(19) United States
(12) Patent Application Publication
(10) Pub. No.: US 2024/0342121 A1
(43) Pub. Date: Oct. 17, 2024

(54) THE USE OF BETA-HYDROXYBUTYRATES FOR THE TREATMENT OR PREVENTION OF ANEURYSMS AND DISSECTIONS

(71) Applicant: Georgia State University Research Foundation, Inc., Atlanta, GA (US)

(72) Inventors: Ming-Hui ZOU, Peachtree Corners, GA (US); Young-Min HAN, Buford, GA (US)

(21) Appl. No.: 18/577,529

(22) PCT Filed: Jul. 9, 2022
(86) PCT No.: PCT/US2022/073582

§ 371 Date: Jan. 8, 2024

Related U.S. Application Data
(60) Provisional application No. 63/220,086, filed on Jul. 9, 2021.

(51) Int. Cl. A61K 31/19; A61K 31/225; A61K 45/06; A61P 9/00

(52) U.S. Cl. CPC A61K 31/19; A61K 31/225; A61K 45/06; A61P 9/00

(57) ABSTRACT

Described herein are methods for treating a subject with an aneurysm or a dissection in a blood vessel comprising administering to the subject an effective amount of a β-hydroxybutyrate. The methods described herein are useful in repairing damage to arteries caused by an aneurysm. Additionally, described herein are methods for reducing or preventing the risk of the formation of aneurysm or dissection in a blood vessel comprising administering to the subject an effective amount of a β-hydroxy butyrate.

### CROSS REFERENCE TO RELATED APPLICATIONS

This application claims the benefit of U.S. Provisional Application No. 63/220,086 filed on Jul. 9, 2021, which is incorporated herein by reference in its entirety.

### BACKGROUND

Abdominal aortic aneurysm (AAA) is a balloon-like swelling in the aortic wall within the abdomen. AAA occurs when the outer aortic diameter is greater than 55 mm with a concomitant increase in the mortality rate, approximately 65% to 85% in case of rupture.¹ AAA occurs most often in people aged 65 and older, suggesting that aging is the major risk factor for AAA development.^{2,3} Research on age-related cardiovascular diseases, including AAA, is urgently demanded due to the rapid increase of the anticipated aging population in the U.S. and European countries.⁴

In the clinic, AAA is a disease that cannot be detected by early diagnosis, and once diagnosed, no distinct treatment methods have been developed other than surgery.⁵ Although there is increasing incidence and mortality associate with AAA, existing medications are only intended to lower blood pressure or delay the expansion of the aorta.⁶ Therefore, to prevent sudden death due to blood vessel rupture, an improved medical treatment procedure is needed. Hence, it is worth developing therapeutic resources for AAA to mitigate the progression of aneurysm formation and prevent vascular rupture by repairing injured aorta.⁷

Thoracic aortic aneurysm (TAA) are most often clinically silent and identified incidentally on an imaging examination. Initial symptoms of TAA are most often secondary to aortic dissection or rupture, potentially fatal conditions associated with TAA. Aneurysms of the thoracic aorta arise by a process known as cystic medial necrosis or medial degeneration, characterized by a decrease in smooth muscle cells, breakdown of elastin fibers, and increased deposition of proteoglycans in the tunica media of the aortic wall. As the aorta dilates, per Laplace's law, aortic wall stress increases proportionally. Ultimately, as medial degeneration progresses and aortic diameter increases, the risk of aortic complications increases.

The initial aneurysm size at time of diagnosis is the best predictor of growth rate and risk of rupture. Studies on the natural history of TAA demonstrate a sharp acceleration or hinge point for the risk of rupture and dissection as the maximum aortic diameter exceeds 5.5 cm. The risk of death, rupture, or dissection is 6.5% per year for TAA greater than 5 cm and 14.1% per year for TAAs greater than 6.0 cm. The average growth rate of TAA greatly exceeds that of normal aorta, averaging 0.10 cm per year (0.07 for ascending and 0.19 for descending).

The natural history of TAA also depends on the underlying etiology and genetics. The majority of TAAs are degenerative, occurring in association with long-standing increases in wall stress and traditional risk factors for atherosclerosis, including age, hypertension, hyperlipidemia, and smoking. TAA of the descending aorta is often associated with atherosclerosis. A proportion of TAA (approximately 5%), particularly in the ascending aorta, is inherited. Although less common, healed aortic dissection, aortic coarctation, and aortitis are also important etiologies of TAA. Aortitis can be secondary to autoimmune diseases (giant cell arteritis, Takayasu arteritis, Bechet's disease, seronegative spondyloarthropathies, or IgG4 related) or infectious diseases (syphilis, mycotic aneurysms from Staphylococcus or Salmonella, and tuberculosis). More than 20% of patients with TAA or dissection report a family history of TAA, supporting a strong genetic predisposition for the disease, most often inherited in an autosomal dominant pattern.

### SUMMARY

Described herein are methods for treating a subject with an aneurysm or a dissection in a blood vessel comprising administering to the subject an effective amount of β-hydroxybutyrate. The methods described herein are useful in repairing damage to arteries caused by an aneurysm. Additionally, described herein are methods for reducing or preventing the risk of the formation of aneurysm or dissection in a blood vessel comprising administering to the subject an effective amount of a β-hydroxybutyrate.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

In the figures descriptions below, KD refers to a ketogenic diet. KE refers to [(3R)-3-hydroxybutyl] (3R)-3-hydroxybutanoate Cas No. 1208313-97-6 and R-BHB or BHB refers to (R)-3-Hydroxybutyrate, CAS No 625-72-9 and S-BHB refers to(S)-3-hydroxybutyrate Cas No 6168-83-8.

FIGS. 1A-1K. (a) Incidence of AAA by AII-infusion for 4 weeks, and proportion of mice recovered from the AAA after post-treatment of KD and KE for 2 weeks or 4 weeks. (b) Representative image of aorta from mice infused with AII, and post-treated with KD for 2 weeks or 4 weeks. (c) External diameter of abdominal aorta from the mice infused with AII, and post-treated with KD and KE for 4 weeks. (d) Comparison of ultrasound images of AII-induced AAA to the identical aorta post-treated with KD or KE. (e) Time-dependent observation of inner diameter after AII infusion (56 days) and post-treatment of KD or KE (28˜56 days after infusion). (f) Relative comparison of AII-induced aneurysm area prior to or post-treatment of KD or KE. (g, h) Hematoxylin and eosin (H&E) staining of abdominal aorta treated with AII, Post-KD or Post-KE. (i) Circulating βHB concentration while AII infusion and KD and KE treatment. (j) Correlation between abdominal aorta diameter and blood β-HB on the mice developed AII-induced AAA. (k) Correlation between abdominal aorta diameter and blood glucose on the mice developed AII-induced AAA.

FIGS. 2A-2L. (a) Cytotoxicity assay of normal smooth muscle cells (NS) and senescent smooth muscle cells (SMCs). Replicative senescent SMCs (RS) and premature senescent SMCs, stimulated by H₂O₂ (HP), arsenic (Ars), and Nutlin 3a (Nut) treatment were treated with βHB, s-βHB, and ABT-737. (b) Flow cytometry analysis of apoptosis by βHB or s-βHB in radiation (10 Gy)-induced senescent SMC. (c) Quantitation of apoptotic cell and senescent cell after treating βHB and s-βHB in radiation-induced senescent SMC. (d) Representative aorta image of AII-infused mice with post-treatment of ABT-737, βHB and s-βHB. (e) External diameter of abdominal aorta infused with AII and post-treated with ABT-737 βHB and s-βHB. (f) SA-βGal activity assay of the whole aorta infused with AII, and post-treated with ABT-737, KD, or KE. (g) Quantitation of senescent area on aorta of AII-infused mice, and post-treated with KD, KE and ABT-737. (h) TUNEL assay on the abdominal aorta of AII infused mice, and treated with ABT-737, βHB, or s-βHB. SM-α Actin (SMA) and DAPI are co-stained after TUNEL reaction. (i) Caspase 3 activity assay on normal SMCs (NC) treated with βHB, or s-βHB, and ABT-737 comparing to normal cells pretreated with Nutlin-3a. (j) Caspase 3 activity assay on senescent SMCs (SNC) treated with βHB, or s-βHB, and ABT-737 compared to senescent cells pretreated with pifithrin-α. (k) Western blot analysis for p53 and puma expression on abdominal aorta tissue from mice treated with AII or KE. (I) Immunohistochemistry staining for puma expression on abdominal aorta infused by AII and post-treated with KE.

FIGS. 3A-3L. (a) Survival rate of AII-infused mice after treated with βHB, s-βHB and ABT-737. (b) H&E staining of aorta from AII-infused mice after I.P. injection of ABT-737, βHB, s-βHB. (c) Verhoeff Van Gieson/EVG staining of abdominal aorta from AII-infused mice post-treated with KD, KE, βHB, s-βHB and ABT-737. (d) Average number of elastic fibers on abdominal aorta presented in FIG. 3c. (e) Western blot analysis of HEXIM1, Calponin, and SM a-Actin for SMC differentiation markers and Oct4, and MMP2 for dedifferentiation markers, and Bax for apoptosis on abdominal aorta tissue form AII-infused mice with post-treatment of KD or KE. (f) Masson's trichrome staining of abdominal aorta from AII-infused mice with/without post-treatment of KE. (g) Western blot analysis of adipose differentiation-associated proteins (Col1A1, RUNX2, Perilipin 1, Oct4) on abdominal PVAT and Thoracic PVAT tissues from the AII infused mice with post-treatment of KE. (h) Pulse Wave Velocity (PWV) measurements on AII-infused mice post-treatment of KD, KE, βHB, and ABT-737. (i) Structural alteration of aortic wall after ketotherapy.

FIGS. 4A-4H. Representative immunofluorescent staining of SM α-Actin (SMA), PDI, vWF, and DAPI on the abdominal aorta tissues from the mice developed with AII-induced AAA, and post-treated with KD, KE and βHB. (b) Representative confocal immunofluorescent staining of fibroblast surface protein (FSP) on the aorta tissues from the mice developed with AII-induced AAA, and post-treated with KD, KE and βHB. (c) Quantification of cells which express SM-α Actin or FSP in the neointima hyperplasia. (d) Quantitation of neointima hyperplasia area on abdominal aorta from the mice which are post-treated with KD and KE. (e) Western blot analysis of Col1A1, PDI, PU.1 expression on abdominal aorta from the mice developed with AII-induced AAA, and post-treated with KD and KE. (f) Picrosirius staining of abdominal aorta from AII-infused mice with/without post-treatment of KD and KE. The images are taken under two different light sources, dark-field images (upper panel) and bright-field (lower panel). (g) Percentage of fibril collagen (bright red color under dark-field) in total collagen (red color under bright-field). (h) Thickness of connective tissues consisting of fibril collagen, shown bright red in FIG. 4F.

FIGS. 5A-5J. (a) Representative image of aorta which developed CaCl₂)-induced AAA, and post-treated with KD, KE, βHB, s-βHB, or ABT-737. KD is provided to mice for 4 weeks, KE is administrated with drinking water, βHB, s-βHB or ABT-737 are IP injected for 2 weeks with 48h intervals. (b) Incidence of AAA by CaCl₂) treatment and proportion of mice recovered from the AAA after post-treatment of KD, KE, βHB and s-βHB for 2 weeks. (c) Time-dependent observation of inner diameter after CaCl₂) treatment and post-treatment of KD or KE (28˜56 days after surgery). (d) Comparison of ultrasound images of CaCl₂)-induced AAA to the identical aorta post-treated with βHB for 2 weeks. (e) H&E staining of aorta from the mice developed with CaCl₂)-induced AAA, and post-treated with KD, KE, βHB, and s-βHB. (f) SA β-Gal activity assay on aorta which developed CaCl₂)-induced AAA, and post-treated with KD, KE, βHB, s-βHB, or ABT-737. (g) Quantitation of senescent area on aorta developed with CaCl₂)-induced AAA, and post-treated with KD, KE, βHB, s-βHB. (h) Verhoeff Van Gieson/EVG staining of abdominal aorta developed with CaCl₂)-induced AAA, post-treated with KD, KE, βHB and s-βHB. (i) Average number of elastic fibers on abdominal aorta presented at FIG. 5H. (j) Measurement of Pulse Wave Velocity (PWV) on CaCl₂)-infused mice with post-treatment of KD, KE, βHB, and s-βHB.

FIGS. 6A-6C show the experimental design of ketotherapy on AAA developed mice.

FIGS. 7A-7J show the metabolic alteration during post-treatment of KD and KE.

FIGS. 8A-8G show the elimination and prevention of senescent cells by βHB.

FIGS. 9A-9J show the amelioration of metabolic excretion of βHB strengthening therapeutic effect.

FIGS. 10A-10J show the suppression of systemic inflammation by ketotherapy.

FIGS. 11A-11J show neointima hyperplasia formation during post-treatment.

FIGS. 12A-12C show (a) multilayered cell mass induced by co-cultivation with senescent SMCs. Oct4 expression in cell mass formation; (b) SA β-galactosidase activity in multilayered cell mass; and (c) time-dependent alteration of Oct4, Sox3, and Klf4 expression of SMCs after SASP stimulation.

FIG. 13 shows the Western blot analysis of Oct4 expression of the abdominal aorta after AII-infusion with post-treatment of KD or KE. Aorta is isolated indicated weeks after AII infusion.

FIGS. 14A-14B show (a) SA β-Gal activity on Oct4 overexpressed SMCs with or without βHB treatment (2 days). Irradiated SMCs were infected by Lentivirus containing Crispr-dCas9-VP64_Oct4 plasmid prior to treating βHB. The arrow indicates localized senescent cell mass. (b) Western blot analysis after lentivirus (Crispr-dCas9-VP64_Oct4) infection into senescent SMCs without βHB treatment for 1 day or 2 days.

FIGS. 15A-15B show immunoblotting of lysine β-hydroxybutyrylation (KβHB) on whole cell lysate (WCL) and fractionated lysate. (b) Immunofluorescent staining of SMA, KβHB, and DAPI on abdominal aorta.

FIGS. 16A-16B show (a) graphical diagram of histone remodeling by lysine β-hydroxybutyrylation (KβHB) that lowers the epigenetic barrier to promote the Oct4-associated reprogramming process of senescent SMCs. (b) Proposed model of lysine β-hydroxybutyrylation (KβHB) unpacking the nucleosomes to facilitate Oct4-dependent reprogramming.

FIGS. 17A-17B show (a) representative immunofluorescent staining of SM α-Actin (SMA), FSP, and DAPI on the abdominal aorta tissues from the mice developed with AII-induced AAA, and post-treated with KD, KE, and βHB. (b) Quantification of cells expressing SM-α Actin FSP in the arterial wall.

FIGS. 18A-18B show (a) measurements of Pulse Wave Velocity (PWV) on AII-infused mice with post-treatment KD, KE. n=13˜53, ## ##P<0.0001, Ctrl versus AII, **P<0.01, AII verses post-treatment. (b) Verhoeff Van Gieson/EVG staining of abdominal aorta from AII-infused mice post-treated with KD, KE.

FIGS. 19A-19D shows that ketone ester (KE) administration reduces the incidence of both thoracic aortic dissection (TAD) and abdominal aortic dissection in mouse model of aortic dissection in vivo. (a) schematic description of aortic dissection model and ketone treatment. Five-week-old C57BL/6 mice were continuously treated with β-aminopropionitrile (BAPN, 3 g/L/day in drinking water) for 28 days. To test the impacts of ketone body, BAPN-treated mice at day 17 to day 31 were given ketone ester (KE, D-BHB 1-3 butanediol monoester) (20 g/L and 50 gL at 5 mL per day) in drinking water. Further, the BAPN-treated mice, with or without ketone treatment, were continuously infused with angiotensin (AngII, 1.44 mg/kg/day) for three days via subcutaneously implanted osmotic pumps). Three days after AngII infusion, the mice were euthanized to assay for aortic dissection. (b) KE significantly reduces the incidence of TAD; (c) KE significantly reduces the incidence of AAD; (d) High dose of KE (50 g/L) significantly increase the survival rate. #p<0.05, BAPN verse BAPN+AngII; †p<0.05 BAPN+AngII verse BAPN+AngII+KE; BAPN (n=10); BAPN+AngII (n=20); BAPN+AngII+KE 20 g/L (n=20); BAPN+AngII+KE 50 g/L (10).

FIG. 20 shows a mechanism of how the compounds described herein restore vascular integrity in AAA.

### DETAILED DESCRIPTION

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “an inert excipient” includes, but are not limited to, mixtures or combinations of two or more such inert excipients, and the like.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

It should be noted that ratios, concentrations, amounts, rates, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; and the number or type of embodiments described in the specification.

Disclosed are the components to be used to prepare the compositions of the invention as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

It is understood that the compositions disclosed herein have certain functions. Disclosed herein are certain structural requirements for performing the disclosed functions, and it is understood that there are a variety of structures that can perform the same function that are related to the disclosed structures, and that these structures will typically achieve the same result.

As used herein, the terms “optional” or “optionally” means that the subsequently described event or circumstance can or cannot occur, and that the description includes instances where said event or circumstance and instances where it does not.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched. The alkyl group can also be substituted or unsubstituted. For example, the alkyl group can be substituted with one or more groups including, but not limited to, alkyl, cycloalkyl, alkoxy, amino, ether, halide, hydroxy, nitro, silyl, sulfo-oxo, or thiol, as described herein. A “lower alkyl” group is an alkyl group containing from one to six (e.g., from one to four) carbon atoms. The term alkyl group can also be a C1 alkyl, C1-C2 alkyl, C1-C3 alkyl, C1-C4 alkyl, C1-C5 alkyl, C1-C6 alkyl, C1-C7 alkyl, C1-C8 alkyl, C1-C9 alkyl, C1-C10 alkyl, and the like up to and including a C1-C24 alkyl.

Throughout the specification “alkyl” is generally used to refer to both unsubstituted alkyl groups and substituted alkyl groups; however, substituted alkyl groups are also specifically referred to herein by identifying the specific substituent(s) on the alkyl group. For example, the term “halogenated alkyl” or “haloalkyl” specifically refers to an alkyl group that is substituted with one or more halide, e.g., fluorine, chlorine, bromine, or iodine. Alternatively, the term “monohaloalkyl” specifically refers to an alkyl group that is substituted with a single halide, e.g. fluorine, chlorine, bromine, or iodine. The term “polyhaloalkyl” specifically refers to an alkyl group that is independently substituted with two or more halides, i.e. each halide substituent need not be the same halide as another halide substituent, nor do the multiple instances of a halide substituent need to be on the same carbon. The term “alkoxyalkyl” specifically refers to an alkyl group that is substituted with one or more alkoxy groups, as described below. The term “aminoalkyl” specifically refers to an alkyl group that is substituted with one or more amino groups. The term “hydroxyalkyl” specifically refers to an alkyl group that is substituted with one or more hydroxy groups. When “alkyl” is used in one instance and a specific term such as “hydroxyalkyl” is used in another, it is not meant to imply that the term “alkyl” does not also refer to specific terms such as “hydroxyalkyl” and the like.

The term “ketone ester” as used herein is a compound possessing at least one ketone group and at least one ester group.

The term “ketone” as used herein is represented by the formula A¹C(O)A², where A¹ and A² can be, independently, an alkyl, cycloalkyl, alkenyl, cycloalkenyl, alkynyl, cycloalkynyl, aryl, or heteroaryl group.

The term “ester” as used herein is represented by the formula —OC(O)A¹ or —C(O)OA¹, where A¹ can be alkyl, cycloalkyl, alkenyl, cycloalkenyl, alkynyl, cycloalkynyl, aryl, or heteroaryl group.

An “aneurysm” is a localized dilation of a blood vessel wall. Aneurysms may be a result of a hereditary condition or an acquired disease.

An “abdominal aortic aneurysm (AAA)” is a permanent, localized dilation of the abdominal aorta. AAA is characterized in most cases by the formation of intraluminal thrombus (ILT), destructive remodeling of structural connective tissue, and chronic adventitial inflammation. Abdominal aortic aneurysms often grow slowly and usually without symptoms, making them difficult to detect.

A “thoracic aortic aneurysm” (TAA) is a weakened area in the upper part of the aorta. The aorta is the major blood vessel that feeds blood to the body. A thoracic aortic aneurysm may also be called thoracic aneurysm and aortic dissection (TAAD) because an aneurysm can lead to a tear in the artery wall (dissection) that can cause life-threatening bleeding.

A “cerebral aneurysm” (CA) is a bulge or ballooning in a blood vessel in the brain. Cerebral aneurysms most commonly occur in the anterior communicating artery, posterior communicating artery, anterior cerebral artery, middle cerebral artery, posterior cerebral artery, internal carotid artery, or the tip of the basilar artery. A brain aneurysm can leak or rupture, causing bleeding into the brain (hemorrhagic stroke). Most often a ruptured brain aneurysm occurs in the space between the brain and the thin tissues covering the brain. This type of hemorrhagic stroke is called a subarachnoid hemorrhage. A ruptured aneurysm quickly becomes life-threatening and requires prompt medical treatment.

A “dissection” is a tear within the wall of a blood vessel, which allows blood to separate the wall layers. By separating a portion of the wall of the artery (a layer of the tunica intima or tunica media), a dissection creates two lumens or passages within the vessel, the native or true lumen, and the “false lumen” created by the new space within the wall of the artery. In one aspect, the dissection is an abdominal aortic dissection or a thoracic aortic dissection. Aortic dissection may be associated with aortic aneurysm or may occur in the absence of or independently of aortic aneurysm.

The term “treat or treatment” as used herein is defined as reducing one or more symptoms of an aneurysm or a dissection in a blood vessel when the subject is administered a β-hydroxybutyrate as described herein when compared to the same symptom(s) in the absence of the administration of the β-hydroxybutyrate to the subject. In one aspect, the β-hydroxybutyrate reduces the size of aneurysmal plaque in the subject when compared to the absence of the administration of the β-hydroxybutyrate to the subject. In another aspect, the β-hydroxybutyrate reduces the inner diameter, exterior diameter, or a combination thereof of the aneurysm in the subject when compared to the absence of the administration of the β-hydroxybutyrate to the subject. In another aspect, the β-hydroxybutyrate recovers the stiffness of the vessel wall of the aneurysm when compared to the absence of the administration of the β-hydroxybutyrate to the subject.

The term “prevent or prevention” as used herein is defined as eliminating or reducing the likelihood of the occurrence of one or more symptoms of an aneurysm or a dissection in a blood vessel when the subject is administered a β-hydroxybutyrate as described herein when compared to the same symptom(s) in the absence of the administration of the β-hydroxybutyrate to the subject. In one aspect, the β-hydroxybutyrate reduces the formation of an aneurysm or dissection in a blood vessel when compared to the absence of the administration of the β-hydroxybutyrate to the subject.

The term “subject” is a human or non-human animal in need of treatment or prevention of an aneurysm or a dissection in a blood vessel. Typically, the subject is a human. A “subject” also refers to for example, a mammal, primate (e.g., human), cows, sheep, goat, horse, dog, cat, rabbit, rat, mice, fish, bird and the like.

The term “effective amount” of a compound means an amount effective, when administered to a subject, to provide a therapeutic benefit such as an amelioration of symptoms or prevention, reduction, or diminution of the disease itself.

The term “pharmaceutically acceptable salts”, as used herein, means salts of the active principal agents which are prepared with acids or bases that are tolerated by a biological system or tolerated by a subject or tolerated by a biological system and tolerated by a subject when administered in a therapeutically effective amount. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include, but are not limited to; sodium, potassium, calcium, ammonium, organic amino, magnesium salt, lithium salt, strontium salt or a similar salt. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include, but are not limited to; those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like.

As used herein, “administering” can refer to an administration that is oral, topical, intravenous, subcutaneous, transcutaneous, transdermal, intramuscular, intra-joint, parenteral, intra-arteriole, intradermal, intraventricular, intraosseous, intraocular, intracranial, intraperitoneal, intralesional, intranasal, intracardiac, intraarticular, intracavernous, intrathecal, intravitreal, intracerebral, and intracerebroventricular, intratympanic, intracochlear, rectal, vaginal, by inhalation, by catheters, stents or via an implanted reservoir or other device that administers, either actively or passively (e.g. by diffusion) a composition the perivascular space and adventitia. For example, a medical device such as a stent can contain a composition or formulation disposed on its surface, which can then dissolve or be otherwise distributed to the surrounding tissue and cells. The term “parenteral” can include subcutaneous, intravenous, intramuscular, intra-articular, intra-synovial, intrasternal, intrathecal, intrahepatic, intralesional, and intracranial injections or infusion techniques. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, “effective amount” can refer to the amount of a disclosed compound or pharmaceutical composition provided herein that is sufficient to effect beneficial or desired biological, emotional, medical, or clinical response of a cell, tissue, system, animal, or human. An effective amount can be administered in one or more administrations, applications, or dosages. The term can also include within its scope amounts effective to enhance or restore to substantially normal physiological function.

As used herein, the term “therapeutically effective amount” refers to an amount that is sufficient to achieve the desired therapeutic result or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors within the knowledge and expertise of the health practitioner and which may be well known in the medical arts. In the case of treating a particular disease or condition, in some instances, the desired response can be inhibiting the progression of the disease or condition. This may involve only slowing the progression of the disease temporarily. However, in other instances, it may be desirable to halt the progression of the disease permanently. This can be monitored by routine diagnostic methods known to one of ordinary skill in the art for any particular disease. The desired response to treatment of the disease or condition also can be delaying the onset or even preventing the onset of the disease or condition.

Throughout the specification and claims, a given chemical formula or name shall encompass all optical and stereoisomers, as well as racemic mixtures where such isomers and mixtures exist, unless otherwise noted.

Unless otherwise specified, temperatures referred to herein are based on atmospheric pressure (i.e. one atmosphere).

### Methods for the Treatment or Prevention of Aneurysms and Dissections

Described herein are methods for treating a subject with an aneurysm, a dissection in a blood vessel, or a combination thereof comprising administering to the subject an effective amount of a β-hydroxybutyrate. The methods described herein are useful in repairing damage to arteries caused by an aneurysm or dissection. Additionally, described herein are methods for reducing or preventing the risk of the formation of aneurysm, a dissection in a blood vessel, or a combination thereof comprising administering to the subject an effective amount of a β-hydroxybutyrate.

Tears or dissections in one or more of the layers of a vessel wall or a ruptured aneurysm are the main complications of aneurysms. A ruptured aneurysm can lead to life-threatening internal bleeding. In general, the larger the aneurysm and the faster the aneurysm grows, the greater the risk of rupture. Aneurysms often are associated with increasing the diameter of vessel walls. The compounds described herein can reduce the inner diameter, exterior diameter, or a combination thereof of the aneurysm in the subject and ultimately reduce the risk of rupture. In other aspects, the compounds described herein can reduce or prevent the incidence of a dissection in a blood vessel. In another aspect, the compounds described herein can reduce the severity of a dissection in a blood vessel.

The vascular cells isolated from the patients with aneurysms such as, for example, abdominal aortic aneurysm have the phenotype features commonly observed in senescent cells. Uncontrolled aortic smooth muscle cell (SMC) growth and migration play a central role in expanding arterial walls and subsequently accumulating senescent cells. Senescence is a cellular stress response triggered by multiple stress signals caused by replicative exhaustion, genotoxic damages (irradiation or chemotherapy), or aberrant oncogene activation (oncogene-induced senescence OIS). Senescent cells arrest stably, produce a complex secretome (known as the senescence-associated secretory phenotype, SASP), and undergo characteristic changes including transcriptional, epigenetic, morphological, and metabolic alterations. Senescent cells can communicate via direct cell-cell contact, cell fusion, through the formation of cytoplasmic bridges, extracellular vesicle (EV) signaling, and through the SASP. Most of the non-cell-autonomous effects of senescent cells have been linked to the SASP. Acute senescence induction protects against cancer and limits fibrosis, but lingering senescent cells drive age-related disorders, such as for example abdominal aortic aneurysm. In one aspect, the compounds described herein remove senescent cells from the vessel of the subject in order to treat or prevent an aneurysm or a dissection in a blood vessel of the subject. In another aspect, the compounds described herein prevents the formation of senescent cells in a vessel of the subject in order to treat or prevent an aneurysm or a dissection in a blood vessel of the subject.

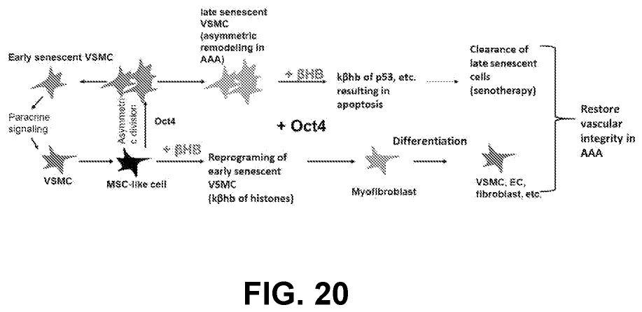

In one aspect, the compounds described herein can convert senescent cells to non-senescent cells. Not wishing to be bound by theory, Oct4 can promote the MSC-like cells for asymmetric divisions, which results in accumulation of late senescent cells that accumulate in AAA areas and form asymmetric lesions. In one aspect, in the presence of OCT4, the compounds described herein (e.g., βHB) can selectively remove late stage senescent cells in the asymmetric site of vascular walls by promoting apoptotic death of these cells. In another aspect, the compounds described herein can promote partial reprogramming of senescent cells through β-hydroxybutyrylation (KβHB) on histone, which enhances the reprogramming functions of Oct4. These reprogrammed senescence SMCs are finally re-differentiated into matured SMCs or myofibroblasts to repair injured aortic walls. FIG. 20 depicts the proposed mechanism described above.

A significant issue associated with aneurysms is that the integrity of the vessel wall of the aneurysm is compromised, which leads to a greater risk of rupture. Vascular smooth muscle cells (VSMC) manifests phenotypic plasticity by changes in the environment. In one aspect, the compounds described herein converts vascular smooth muscle cells into myofibroblasts. Not wishing to be bound by theory, the compounds described herein can reprogram (i.e., re-differentiate) senescent VSMCs into myofibroblasts, resulting in improved tissue repair capacity, as fibroblast-induced ECM remodeling is essential for repairing injured tissues. For example, histone β-hydroxybutyrylation produced by the administration of the compounds described herein can enhance the transcriptional activity Oct4 to promote reprogramming of senescent smooth muscle cells through synergistic effects of chromatin remodeling and Oct4 activation, which results in re-differentiation into matured VSMCs and myofibroblast. In one aspect, the compounds and methods described herein can enhance or promote the restoration of healthy phenotypes of VSMCs in a vessel in which there is pathology arising from senescent or otherwise dysfunctional VSMCs. Thus, the compounds and methods described herein can promote tissue regeneration that improves the repair capacity of injured tissue.

In one aspect, the compounds and methods described herein can recover the stiffness or integrity of the vessel wall of an aneurysm or dissection to an approximate state prior to the formation of the aneurysm or dissection. Not wishing to be bound by theory, fibroblasts play an important role in vascular regeneration based on their high-plasticity and rejuvenation ability. In one aspect, redifferentiated fibroblasts can form neointima hyperplasia to prevent aortic dissection when the subject is administered a compound described herein. In another aspect, administration of the compounds described herein can reduce neointima hyperplasia and simultaneously promoted regeneration of tunica media smooth muscle cells (SMCs), elastic lamina and perivascular connective tissue. This result reveals the possibility of trans-differentiation of fibroblast to endothelial cells EC and SMC. Thus, the fibroblast transition to endothelial and smooth muscle cells is a unique cellular process that safely regenerates the vessel wall, which in turn can delay or prevent the burst of the aneurysm or increased dissection in the vessel.

In another aspect, the compounds and methods described herein can reduce the size or volume of aneurysmal plaque in the subject. Not wishing to be bound by theory, clearance of senescent cells accumulated significantly during aneurysm development is required to reduce the volume of aneurysm plaque. In one aspect, the compounds described herein possess a senotherapeutic potential to clear senescent plaque via regulating p53. Thus, in another aspect, the compounds and methods described herein can treat or preventing atherosclerosis in a subject

In one aspect, the subject is predisposed to the development of an aneurysm or a dissection. In one aspect, the subject is screened for a genetic mutation that predisposes the subject to an aneurysm, a dissection in a blood vessel, or a combination thereof. In one aspect, the subject has a mutated gene that encodes proteins involved in vascular smooth muscle cell contraction and adhesion to the extracellular matrix (ECM), transforming growth factor)-β signaling pathway, or smooth muscle cell metabolism. In another aspect, the subject has a mutated gene, wherein the gene includes FBN1, lysyl oxidase (LOX), smooth muscle myosin heavy chain 11 (MYH11), smooth muscle α-actin 2 (ACTA2), myosin light chain kinase (MYLK), protein kinase cGMP-dependent type 1 (PRKG1), α-1 procollagen, type III (COL3A1), TGF-β receptor type II (TGFBR2), TGF-β receptor type I (TGFBR1), TGF-β2 (TGFB2), mothers against decapentaplegic drosophila homolog 3 (SMAD3), α-1 procollagen, type I (COL1A1), α-2 procollagen, type I (COL1A2), mediator complex subunit 12 (MED12), mothers against decapentaplegic drosophila homolog 4 (SMAD4), procollagen-lysine, 2-oxoglutarate 5-dioxygenase 3 (PLOD3), endoglin (ENG), activin A receptor like Type 1 (ACVRL1), neurofibromatosis type 1 (NF1), or any combination thereof. In one aspect, the methods disclosed in Hannuksela et al., Aorta, 2015, 3 (1), 1-8 (doi:10.12945/j.aorta.2015.14-052), which is incorporated by reference, can be used in the genetic screening of mutated genes associated with aneurysm and dissections.

In another aspect, the subject can be screened for the presence of one or more biomarker that is indicative of the presence of an aneurysm or dissection or if the subject is predisposed to the formation of an aneurysm or dissection. In one aspect, the biomarker is an elevated level of amount of 3-hydroxyanthranilic acid (3-HAA). In one aspect, the method involves
(a) determining the level of 3-hydroxyanthranilic acid (3-HAA) present in a sample from the subject;
(b) comparing the subject's level of 3-HAA to a range of standardized levels of 3-HAA derived from individuals without an aneurysm or dissection in a blood vessel (“normal range”); and
(c) administering the compound to subject if the subject's levels of 3-HAA is greater than the normal range.

The methods disclosed in WO2019/006384, which is incorporated by reference, can be used to determine the amount of 3-hydroxyanthranilic acid (3-HAA) in a subject.

In one aspect, the subject has a genetic disorder associated with an increased risk of aneurysm development. In one aspect, the subject has a genetic disorder selected from Marfan syndrome, Loeys-Dietz syndrome, Ehlers-Danlos syndrome, Familial Thoracic Aortic Aneurysm and Dissection, Mitral valve, myopia, Aorta, Skin and Skeletal (MASS) syndrome, Beals syndrome, aneurysms-osteoarthritis syndrome, Shprintzen-Goldberg syndrome, cutis laxa syndrome, aortic valve disease, arterial tortuosity syndrome, X-linked Alport syndrome, Turner syndrome, and Bicuspid Aortic Valve syndrome.

### Compounds

In one aspect, the compound used in the methods described herein has structure I or the pharmaceutically acceptable salt thereof:

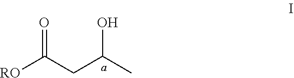

wherein R is hydrogen, an alkyl group or a hydroxyalkyl group, and
the stereochemistry at carbon a is substantially R, substantially S, or racemic,
or wherein the compound is a dimer or trimer of the compound of structure I.

In one aspect, the stereochemistry at carbon a is substantially R or S, where the term “substantially” refers to greater than 80%, greater than 85%, greater than 90%, greater than 95%, greater than 99%, or 100%.

In one aspect, R in structure I is hydrogen. In another aspect, R in structure I is hydrogen and the stereochemistry at carbon a is substantially S. In another aspect, R in structure I is a C₁ to C₁₀ hydroxyalkyl group. In another aspect, R in structure I has the structure II

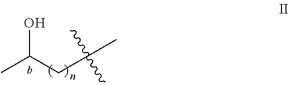

wherein n is an integer from 1 to 5 and the stereochemistry at carbon b is substantially R, substantially S, or racemic.

Compounds having the structure I where R is not hydrogen can be synthesized using techniques known in the art. For example, the carboxylic acid of β-hydroxybutyrate can be reacted with a base such as, for example, triethylamine and an alkyl halide, for example with methyl iodide, benzyl iodide, cyclopentyl iodide or alkyl triflate. They also can be prepared by reaction of β-hydroxybutyrate with an acid such as hydrochloric acid and an alcohol ROH such as ethanol or methanol.

In one aspect, the compound administered to the subject is (R)-β-hydroxybutyrate, (S)-β-hydroxybutyrate, (R)-3-hydroxybutyl-(R)-3-hydroxybutanoate, (R)-3-hydroxybutyl-(S)-3-(S)-3-hydroxybutyl-(S)-3-hydroxybutanoate, (S)-3-hydroxybutyl-(R)-3-hydroxybutanoate, hydroxybutanoate, or the pharmaceutically acceptable salt thereof.

In another aspect, dimers and trimers of a compound having structure I can be used herein. In one aspect, the dimer is the reaction product between a diol and the compound of structure I (e.g., β-hydroxybutyrate). Examples of a diol include a C₂ to C₆ diol such as ethylene glycol or propylene glycol. In another aspect, the trimer is the reaction product between triol and the compound of structure I. Examples of a triol include a C₂ to C₆ triol such as glycerol.

In one aspect, the compound has structure III or the pharmaceutically acceptable salt thereof

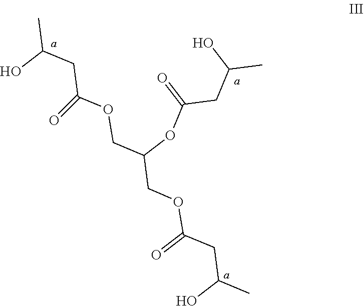 [SMILES: CC(O)CC(=O)OCC(COC(=O)CC(C)O)OC(=O)CC(C)O]

wherein the stereochemistry at carbon a is substantially S or racemic, where the compound is a trimer produced by the reaction between glycerol and β-hydroxybutyrate.

In one aspect, the compounds disclosed in US patent and Publication Nos. U.S. Pat. No. 10,821,062; 10,478,415; 2019/0014798; 2020/0140371; 2020/0222353; 10,051,880; and 11,230,722, which are incorporated by reference, can be used as the compound having the structure I. The synthetic methods disclosed in Budin et al, Bioorganic Chemistry, 80, (2018), pg. 560-564, which are incorporated by reference, can be used to make the compounds described herein.

### Additional Treatment

In addition to the administration of the compounds described herein, the subject can undergo additional or concurrent treatment to treat or prevent an aneurysm. In one aspect, the subject can undergo ketotherapy in conjunction with the administration of a compound described herein. Ketotherapy is a well-known dietary intervention that alleviates symptoms of age-associated diseases, such as neurodegenerative disorders, metabolic syndrome, and cancers. In one aspect, the subject can undergo ketogenic diet (KD) and/or take a ketone ester prior to and/or during the administration of the compounds described herein.

In other aspects, the subject can be administered one or more additional therapeutic agents in conjunction with the administration of a compound described herein. In one aspect, the subject is furthered administered an effective amount of a KYNU inhibitor, a KMO inhibitor, a 3-HAO upregulator, or acipimox, or a pharmaceutically acceptable salt thereof. Examples of such therapeutics are provided in US Publication No. 2020/0138810, which is incorporated by reference for all teachings related to these therapeutics.

In one aspect, in addition to the administration of the compounds described herein, paeonol can be administered to the subject. In another aspect, a senolytic such as, for example, fisetin and quercetin can be administered to the subject in combination with the compounds described herein. In another aspect, a senolytic such as, for example, a NOX4 or NOX4 subtype inhibitors can be administered to the subject in combination with the compounds described herein.

In another aspect, PDCE1 inhibitors may be used in combination with the methods described herein. In another aspect, delivery of the anticancer drug lenvatinib via percutaneous balloon angioplasty can be used in combination with the methods described herein. Administration of lenvatinib in combination with the administration of the a compound described herein be used to prevent re-establishment or renewed progression of AAA in a patient treated for AAA with lenvatinib.

In addition to administering the compounds described herein, other supplements that elevate βHB can be administered. In one aspect, medium chain triglyceric acids such as caproic acid, caprylic acid, capric acid, or lauric acid can be administered. In another aspect, pharmaceutical strategies that deplete glucose are also known to elevate βHB. The drug class known as SGLT2 inhibitors display this activity. SGLT2 inhibitors such as canagliflozin, dapagliflozin, empagliflozin and ertugliflozin are known. In one aspect, low dose SGLT2 can complement the methods described herein by allowing elevated levels of circulating βHB to be achieved by administering fewer grams of βHB to the patient in need of AAA therapy. In this context low dose SGLT2 means doses that are between 5% and 50% of doses given for other indications.

In one aspect, the methods described herein provide βHB levels in the range of ˜1.3 mM and maintained there 15+ hours per week. Inexpensive home monitors for βHB levels are available (https://www.dietdoctor.com/low-carb/keto/best-ketone-meter). In one aspect, a patient in need of treatment for AAA or TAA is prescribed a therapeutic dose of a compounds as described herein alone or in combination with a second bioactive as discussed above. The patient then uses a ketone meter as recommended and determines and reports βHB mM achieved following therapy for a period of days so that the dose may be adjusted to achieve the desired circulating levels of βHB.

In another aspect, endovascular treatment can be performed in combination with the methods described herein. For example, endovascular treatment of abdominal aortic aneurysms (EVAR) is a less invasive surgical procedure than so called “open repair.” EVAR is done percutaneously (through the skin). It usually involves two small incisions made in the groin to expose the femoral arteries. A synthetic graft and stents are fed through these arteries with catheters and guidewires until the graft is positioned correctly at the top and bottom of the defective portion of the aorta (AAA). Removal of the sheath with or without balloon expansion allows barbs or other fixing devices to attach to the artery wall and hold the graft firmly in place, allowing blood to pass through it and remove pressure from the weakened aortic wall.

### Pharmaceutical Compositions

In various aspects, the present disclosure relates to pharmaceutical compositions comprising a therapeutically effective amount of at least one disclosed compound, at least one product of a disclosed method, or a pharmaceutically acceptable salt thereof. As used herein, “pharmaceutically-acceptable carriers” means one or more of a pharmaceutically acceptable diluents, preservatives, antioxidants, solubilizers, emulsifiers, coloring agents, releasing agents, coating agents, sweetening, flavoring and perfuming agents, and adjuvants. The disclosed pharmaceutical compositions can be conveniently presented in unit dosage form and prepared by any of the methods well known in the art of pharmacy and pharmaceutical sciences.

The amount of the compound described herein that can be administered to the subject can vary. In one aspect, the half-life of the compounds described herein can vary, which will in turn can vary the dosage administered to the subject. In one aspect, when the stereochemistry at carbon a in structure I is substantially S, the half-life of the compound upon administration to the subject is about two times to about 10 times greater than the half-life of the same compound but where the stereochemistry at carbon a in structure I is substantially R. In another aspect, the half-life of the compound upon administration to the subject is about two times, about three times, about four times, about five times, about six times, about seven times, about eight times, about nine times, or about 10 times greater than the half-life of the same compound but where the stereochemistry at carbon a in structure I is substantially R, where any value can be a lower and upper endpoint of a range (e.g., about 4 times to about 6 times).

Not wishing to be bound by theory, [(3R)-3-hydroxybutyl] (3R)-3-hydroxybutanoate (KE) undergoes complete enzymatic hydrolysis to release R-βHB and R-butane-1,3-diol. R-1,3-butanediol is further metabolized in the liver and blood to produce R-βHB. βHB in the liver is metabolized to AcAc and acetone (see Adrian Soto-Mota et al., “Safety and Tolerability of Sustained Exogenous Ketosis Using Ketone Monoester Drinks for 28 Days in Healthy Adults,” Regulatory Toxicology and Pharmacology: RTP 109 (December 2019): 104506, https://doi.org/10.1016/j.yrtph.2019.104506.

In one aspect, S-βHB R-butandiol may be administered to a patient in need of AAA or TAA therapy. This would be expected to rapidly release S-βHB (esterases) and R-butane-1,3-diol. R-1,3-butanediol would be further metabolized in the liver and blood to produce R-βHB as discussed above. This prodrug may have advantages in AAA, TAA or other indications in which both R-βHB and S-βHB activity may be beneficial. S-βHB R-butandiol would also be expected to have a half more suitable for a pharmaceutical than KE (R-βHB-R-butandiol).

In one aspect, the dosage of the compounds described herein administered to the subject is from about 0.05 g/kg/day to about 0.20 g/kg/day, or about 0.05 g/kg/day, 0.06 g/kg/day, 0.07 g/kg/day, 0.08 g/kg/day, 0.09 g/kg/day, 0.10 g/kg/day, 0.11 g/kg/day, 0.05 g/kg/day, 0.12 g/kg/day, 0.13 g/kg/day, 0.14 g/kg/day, 0.15 g/kg/day, 0.17 g/kg/day, 0.18 g/kg/day, 0.19 g/kg/day, or 0.20 g/kg/day, where any value can be a lower and upper endpoint of a range (e.g., about 0.08 g/kg/day to about 0.15 g/kg/day).

the dosage of the compound described herein administered to the subject is from about 10 mg/kg/day to about 100 mg/kg/day, or about 10 mg/kg/day, 20 mg/kg/day, 30 mg/kg/day, 40 mg/kg/day, 50 mg/kg/day, 60 mg/kg/day, 70 mg/kg/day, 80 mg/kg/day, 90 mg/kg/day, or 100 mg/kg/day, where any value can be a lower and upper endpoint of a range (e.g., about 40 mg/kg/day to about 60 mg/kg/day).

In a further aspect, the disclosed pharmaceutical compositions comprise a therapeutically effective amount of at least one disclosed compound, at least one product of a disclosed method, or a pharmaceutically acceptable salt thereof as an active ingredient, a pharmaceutically acceptable carrier, optionally one or more other therapeutic agent, and optionally one or more adjuvant. The disclosed pharmaceutical compositions include those suitable for oral, rectal, topical, pulmonary, nasal, and parenteral administration, although the most suitable route in any given case will depend on the particular host, and nature and severity of the conditions for which the active ingredient is being administered. In a further aspect, the disclosed pharmaceutical composition can be formulated to allow administration orally, nasally, via inhalation, parenterally, paracancerally, transmucosally, transdermally, intramuscularly, intravenously, intradermally, subcutaneously, intraperitoneally, intraventricularly, intracranially and intratumorally.

As used herein, “parenteral administration” includes administration by bolus injection or infusion, as well as administration by intravenous, intramuscular, intraarterial, intrathecal, intracapsular, intraorbital, intracardiac, intradermal, intraperitoneal, transtracheal, subcutaneous, subcuticular, intraarticular, subcapsular subarachnoid, intraspinal, epidural and intrasternal injection and infusion.

In various aspects, the present disclosure also relates to a pharmaceutical composition comprising a pharmaceutically acceptable carrier or diluent and, as active ingredient, a therapeutically effective amount of a disclosed compound, a product of a disclosed method of making, a pharmaceutically acceptable salt, a hydrate thereof, a solvate thereof, a polymorph thereof, or a stereochemically isomeric form thereof. In a further aspect, a disclosed compound, a product of a disclosed method of making, a pharmaceutically acceptable salt, a hydrate thereof, a solvate thereof, a polymorph thereof, or a stereochemically isomeric form thereof, or any subgroup or combination thereof may be formulated into various pharmaceutical forms for administration purposes.

Pharmaceutically acceptable salts can be prepared from pharmaceutically acceptable non-toxic bases or acids. For therapeutic use, salts of the disclosed compounds are those wherein the counter ion is pharmaceutically acceptable. However, salts of acids and bases which are non-pharmaceutically acceptable may also find use, for example, in the preparation or purification of a pharmaceutically acceptable compound. All salts, whether pharmaceutically acceptable or not, are contemplated by the present disclosure. Pharmaceutically acceptable acid and base addition salts are meant to comprise the therapeutically active non-toxic acid and base addition salt forms which the disclosed compounds are able to form.

In practice, the compounds of the present disclosure, or pharmaceutically acceptable salts thereof, of the present disclosure can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present disclosure can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the present disclosure, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

It is especially advantageous to formulate the aforementioned pharmaceutical compositions in unit dosage form for ease of administration and uniformity of dosage. The term “unit dosage form,” as used herein, refers to physically discrete units suitable as unitary dosages, each unit containing a predetermined quantity of active ingredient calculated to produce the desired therapeutic effect in association with the required pharmaceutical carrier. That is, a “unit dosage form” is taken to mean a single dose wherein all active and inactive ingredients are combined in a suitable system, such that the patient or person administering the drug to the patient can open a single container or package with the entire dose contained therein, and does not have to mix any components together from two or more containers or packages. Typical examples of unit dosage forms are tablets (including scored or coated tablets), capsules or pills for oral administration; single dose vials for injectable solutions or suspension; suppositories for rectal administration; powder packets; wafers; and segregated multiples thereof. This list of unit dosage forms is not intended to be limiting in any way, but merely to represent typical examples of unit dosage forms.

The pharmaceutical compositions disclosed herein comprise a compound of the present disclosure (or pharmaceutically acceptable salts thereof) as an active ingredient, a pharmaceutically acceptable carrier, and optionally one or more additional therapeutic agents. In various aspects, the disclosed pharmaceutical compositions can include a pharmaceutically acceptable carrier and a disclosed compound, or a pharmaceutically acceptable salt thereof. In a further aspect, a disclosed compound, or pharmaceutically acceptable salt thereof, can also be included in a pharmaceutical composition in combination with one or more other therapeutically active compounds. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The compounds described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for oral, rectal, topical, intravenous injection or parenteral administration. Carriers include solids or liquids, and the type of carrier is chosen based on the type of administration being used. The compounds may be administered as a dosage that has a known quantity of the compound.

Because of the ease in administration, oral administration can be a preferred dosage form, and tablets and capsules represent the most advantageous oral dosage unit forms in which case solid pharmaceutical carriers are obviously employed. However, other dosage forms may be suitable depending upon clinical population (e.g., age and severity of clinical condition), solubility properties of the specific disclosed compound used, and the like. Accordingly, the disclosed compounds can be used in oral dosage forms such as pills, powders, granules, elixirs, tinctures, suspensions, syrups, and emulsions. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

The disclosed pharmaceutical compositions in an oral dosage form can comprise one or more pharmaceutical excipient and/or additive. Non-limiting examples of suitable excipients and additives include gelatin, natural sugars such as raw sugar or lactose, lecithin, pectin, starches (for example corn starch or amylose), dextran, polyvinyl pyrrolidone, polyvinyl acetate, gum arabic, alginic acid, tylose, talcum, lycopodium, silica gel (for example colloidal), cellulose, cellulose derivatives (for example cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate), fatty acids as well as magnesium, calcium or aluminum salts of fatty acids with 12 to 22 carbon atoms, in particular saturated (for example stearates), emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C₁₂H₂₄O₂ to C₁₈H₃₆O₂ and their mixtures, it being possible for the glycerol hydroxy groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentacrythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with C1-C12-alcohols, dimethylacetamide, lactamides, lactates, ethylcarbonates, silicones (in particular medium-viscous polydimethyl siloxanes), calcium carbonate, sodium carbonate, calcium phosphate, sodium phosphate, magnesium carbonate and the like.

Other auxiliary substances useful in preparing an oral dosage form are those which cause disintegration (so-called disintegrants), such as: cross-linked polyvinyl pyrrolidone, sodium carboxymethyl starch, sodium carboxymethyl cellulose or microcrystalline cellulose. Conventional coating substances may also be used to produce the oral dosage form. Those that may for example be considered are: polymerizates as well as copolymerizates of acrylic acid and/or methacrylic acid and/or their esters; copolymerizates of acrylic and methacrylic acid esters with a lower ammonium group content (for example Eudragit® RS), copolymerizates of acrylic and methacrylic acid esters and trimethyl ammonium methacrylate (for example Eudragit® RL); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate, -phthalate succinate as well as methyl cellulose phthalic acid half ester; zein; ethyl cellulose as well as ethyl cellulose succinate; shellac, gluten; ethylcarboxyethyl cellulose; ethacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine.

Plasticizing agents that may be considered as coating substances in the disclosed oral dosage forms are: citric and tartaric acid esters (acetyl-triethyl citrate, acetyl tributyl-, tributyl-, triethyl-citrate); glycerol and glycerol esters (glycerol diacetate, -triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and diburylsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates such as Polysorbar 50); sorbitan monooleate.

Moreover, suitable binders, lubricants, disintegrating agents, coloring agents, flavoring agents, flow-inducing agents, and melting agents may be included as carriers. The pharmaceutical carrier employed can be, for example, a solid, liquid, or gas. Examples of solid carriers include, but are not limited to, lactose, terra alba, sucrose, glucose, methylcellulose, dicalcium phosphate, calcium sulfate, mannitol, sorbitol talc, starch, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In various aspects, a binder can include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. In a further aspect, a disintegrator can include, for example, starch, methyl cellulose, agar, bentonite, xanthan gum, and the like.

In various aspects, an oral dosage form, such as a solid dosage form, can comprise a disclosed compound that is attached to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period.

A tablet containing a disclosed compound can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In various aspects, a solid oral dosage form, such as a tablet, can be coated with an enteric coating to prevent ready decomposition in the stomach. In various aspects, enteric coating agents include, but are not limited to, hydroxypropylmethylcellulose phthalate, methacrylic acid-methacrylic acid ester copolymer, polyvinyl acetate-phthalate and cellulose acetate phthalate. Akihiko Hasegawa “Application of solid dispersions of Nifedipine with enteric coating agent to prepare a sustained-release dosage form” Chem. Pharm. Bull. 33:1615-1619 (1985). Various enteric coating materials may be selected on the basis of testing to achieve an enteric coated dosage form designed ab initio to have a preferable combination of dissolution time, coating thicknesses and diametral crushing strength (e.g., see S. C. Porter et al. “The Properties of Enteric Tablet Coatings Made from Polyvinyl Acetate-phthalate and Cellulose acetate Phthalate”, J. Pharm. Pharmacol. 22:42p (1970)). In a further aspect, the enteric coating may comprise hydroxypropyl-methylcellulose phthalate, methacrylic acid-methacrylic acid ester copolymer, polyvinyl acetate-phthalate and cellulose acetate phthalate.

In various aspects, an oral dosage form can be a solid dispersion with a water soluble or a water insoluble carrier. Examples of water soluble or water insoluble carrier include, but are not limited to, polyethylene glycol, polyvinylpyrrolidone, hydroxypropylmethyl-cellulose, phosphatidylcholine, polyoxyethylene hydrogenated castor oil, hydroxypropylmethylcellulose phthalate, carboxymethylethylcellulose, or hydroxypropylmethylcellulose, ethyl cellulose, or stearic acid.

In various aspects, an oral dosage form can be in a liquid dosage form, including those that are ingested, or alternatively, administered as a mouth wash or gargle. For example, a liquid dosage form can include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the present disclosure may also be in the form of oil-in-water emulsions, which may also contain excipients such as sweetening and flavoring agents.

For the preparation of solutions or suspensions it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulfoxide, triglycerides and the like.

In the case of a liquid dosage form such as a drinkable solutions, the following substances may be used as stabilizers or solubilizers: lower aliphatic mono- and multivalent alcohols with 2-4 carbon atoms, such as ethanol, n-propanol, glycerol, polyethylene glycols with molecular weights between 200-600 (for example 1 to 40% aqueous solution), diethylene glycol monoethyl ether, 1,2-propylene glycol, organic amides, for example amides of aliphatic C1-C6-carboxylic acids with ammonia or primary, secondary or tertiary C1-C4-amines or C1-C4-hydroxy amines such as urea, urethane, acetamide, N-methyl acetamide, N, N-diethyl acetamide, N, N-dimethyl acetamide, lower aliphatic amines and diamines with 2-6 carbon atoms, such as ethylene diamine, hydroxyethyl theophylline, tromethamine (for example as 0.1 to 20% aqueous solution), aliphatic amino acids.

In preparing the disclosed liquid dosage form can comprise solubilizers and emulsifiers such as the following non-limiting examples can be used: polyvinyl pyrrolidone, sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethyl) imidazolidone-(2). In this context, polyoxyethylated means that the substances in question contain polyoxyethylene chains, the degree of polymerization of which generally lies between 2 and 40 and in particular between 10 and 20. Polyoxyethylated substances of this kind may for example be obtained by reaction of hydroxyl group-containing compounds (for example mono- or diglycerides or unsaturated compounds such as those containing oleic acid radicals) with ethylene oxide (for example 40 Mol ethylene oxide per 1 Mol glyceride). Examples of oleotriglycerides are olive oil, peanut oil, castor oil, sesame oil, cottonseed oil, corn oil. See also Dr. H. P. Fiedler “Lexikon der Hillsstoffe für Pharmazie, Kostnetik und angrenzende Gebiete” 1971, pages 191-195.

In various aspects, a liquid dosage form can further comprise preservatives, stabilizers, buffer substances, flavor correcting agents, sweeteners, colorants, antioxidants and complex formers and the like. Complex formers which may be for example be considered are: chelate formers such as ethylene diamine retrascetic acid, nitrilotriacetic acid, diethylene triamine pentacetic acid and their salts.

It may optionally be necessary to stabilize a liquid dosage form with physiologically acceptable bases or buffers to a pH range of approximately 6 to 9. Preference may be given to as neutral or weakly basic a pH value as possible (up to pH 8).

In order to enhance the solubility and/or the stability of a disclosed compound in a disclosed liquid dosage form, a parenteral injection form, or an intravenous injectable form, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also co-solvents such as alcohols may improve the solubility and/or the stability of the compounds according to the present disclosure in pharmaceutical compositions.

In various aspects, a disclosed liquid dosage form, a parenteral injection form, or an intravenous injectable form can further comprise liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Pharmaceutical compositions of the present disclosure suitable injection, such as parenteral administration, such as intravenous, intramuscular, or subcutaneous administration. Pharmaceutical compositions for injection can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. In some aspects, a disclosed parenteral formulation can comprise about 0.01-0.1 M, e.g. about 0.05 M, phosphate buffer. In a further aspect, a disclosed parenteral formulation can comprise about 0.9% saline.

In various aspects, a disclosed parenteral pharmaceutical composition can comprise pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions or suspensions, including saline and buffered media. Parenteral vehicles can include mannitol, normal serum albumin, sodium chloride solution, Ringer's dextrose, dextrose and sodium chloride, lactated Ringer's and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, collating agents, inert gases and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

In addition to the pharmaceutical compositions described herein above, the disclosed compounds can also be formulated as a depot preparation. Such long-acting formulations can be administered by implantation (e.g., subcutaneously or intramuscularly) or by intramuscular injection. Thus, for example, the compounds can be formulated with suitable polymeric or hydrophobic materials (e.g., as an emulsion in an acceptable oil) or ion exchange resins, or as sparingly soluble derivatives, e.g., as a sparingly soluble salt.

Pharmaceutical compositions of the present disclosure can be in a form suitable for topical administration. As used herein, the phrase “topical application” means administration onto a biological surface, whereby the biological surface includes, for example, a skin area (e.g., hands, forearms, elbows, legs, face, nails, anus and genital areas) or a mucosal membrane. By selecting the appropriate carrier and optionally other ingredients that can be included in the composition, as is detailed herein below, the compositions of the present invention may be formulated into any form typically employed for topical application. A topical pharmaceutical composition can be in a form of a cream, an ointment, a paste, a gel, a lotion, milk, a suspension, an aerosol, a spray, foam, a dusting powder, a pad, and a patch. Further, the compositions can be in a form suitable for use in transdermal devices. These formulations can be prepared, utilizing a compound of the present disclosure, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment.

Ointments are semisolid preparations, typically based on petrolatum or petroleum derivatives. The specific ointment base to be used is one that provides for optimum delivery for the active agent chosen for a given formulation, and, preferably, provides for other desired characteristics as well (e.g., emollience). As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

Lotions are preparations that are to be applied to the skin surface without friction. Lotions are typically liquid or semiliquid preparations in which solid particles, including the active agent, are present in a water or alcohol base. Lotions are typically preferred for treating large body areas, due to the ease of applying a more fluid composition. Lotions are typically suspensions of solids, and oftentimes comprise a liquid oily emulsion of the oil-in-water type. It is generally necessary that the insoluble matter in a lotion be finely divided. Lotions typically contain suspending agents to produce better dispersions as well as compounds useful for localizing and holding the active agent in contact with the skin, such as methylcellulose, sodium carboxymethyl-cellulose, and the like.

Creams are viscous liquids or semisolid emulsions, either oil-in-water or water-in-oil. Cream bases are typically water-washable, and contain an oil phase, an emulsifier and an aqueous phase. The oil phase, also called the “internal” phase, is generally comprised of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic or amphoteric surfactant. Reference may be made to Remington: The Science and Practice of Pharmacy, supra, for further information.

Pastes are semisolid dosage forms in which the bioactive agent is suspended in a suitable base. Depending on the nature of the base, pastes are divided between fatty pastes or those made from a single-phase aqueous gel. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; modified cellulose, such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the active agent can be dissolved. Upon delivery to the skin, the carrier evaporates, leaving concentrated active agent at the site of administration.

Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or aqueous alkanolic, but are typically formulated with high alcohol content which, upon application to the skin of a user, quickly evaporates, driving the active ingredient through the upper skin layers to the site of treatment.

Skin patches typically comprise a backing, to which a reservoir containing the active agent is attached. The reservoir can be, for example, a pad in which the active agent or composition is dispersed or soaked, or a liquid reservoir. Patches typically further include a frontal water permeable adhesive, which adheres and secures the device to the treated region. Silicone rubbers with self-adhesiveness can alternatively be used. In both cases, a protective permeable layer can be used to protect the adhesive side of the patch prior to its use. Skin patches may further comprise a removable cover, which serves for protecting it upon storage.

Examples of patch configuration which can be utilized with the present invention include a single-layer or multi-layer drug-in-adhesive systems which are characterized by the inclusion of the drug directly within the skin-contacting adhesive. In such a transdermal patch design, the adhesive not only serves to affix the patch to the skin, but also serves as the formulation foundation, containing the drug and all the excipients under a single backing film. In the multi-layer drug-in-adhesive patch a membrane is disposed between two distinct drug-in-adhesive layers or multiple drug-in-adhesive layers are incorporated under a single backing film.

Examples of pharmaceutically acceptable carriers that are suitable for pharmaceutical compositions for topical applications include carrier materials that are well-known for use in the cosmetic and medical arts as bases for e.g., emulsions, creams, aqueous solutions, oils, ointments, pastes, gels, lotions, milks, foams, suspensions, aerosols and the like, depending on the final form of the composition. Representative examples of suitable carriers according to the present invention therefore include, without limitation, water, liquid alcohols, liquid glycols, liquid polyalkylene glycols, liquid esters, liquid amides, liquid protein hydrolysates, liquid alkylated protein hydrolysates, liquid lanolin and lanolin derivatives, and like materials commonly employed in cosmetic and medicinal compositions. Other suitable carriers according to the present invention include, without limitation, alcohols, such as, for example, monohydric and polyhydric alcohols, e.g., ethanol, isopropanol, glycerol, sorbitol, 2-methoxyethanol, diethyleneglycol, ethylene glycol, hexyleneglycol, mannitol, and propylene glycol; ethers such as diethyl or dipropyl ether; polyethylene glycols and methoxypolyoxyethylenes (carbowaxes having molecular weight ranging from 200 to 20,000); polyoxyethylene glycerols, polyoxyethylene sorbitols, stearoyl diacetin, and the like.

Topical compositions of the present disclosure can, if desired, be presented in a pack or dispenser device, such as an FDA-approved kit, which may contain one or more unit dosage forms containing the active ingredient. The dispenser device may, for example, comprise a tube. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising the topical composition of the invention formulated in a pharmaceutically acceptable carrier may also be prepared, placed in an appropriate container, and labeled for treatment of an indicated condition.

Another patch system configuration which can be used by the present invention is a reservoir transdermal system design which is characterized by the inclusion of a liquid compartment containing a drug solution or suspension separated from the release liner by a semi-permeable membrane and adhesive. The adhesive component of this patch system can either be incorporated as a continuous layer between the membrane and the release liner or in a concentric configuration around the membrane. Yet another patch system configuration which can be utilized by the present invention is a matrix system design which is characterized by the inclusion of a semisolid matrix containing a drug solution or suspension which is in direct contact with the release liner. The component responsible for skin adhesion is incorporated in an overlay and forms a concentric configuration around the semisolid matrix.

Pharmaceutical compositions of the present disclosure can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

Pharmaceutical compositions containing a compound of the present disclosure, and/or pharmaceutically acceptable salts thereof, can also be prepared in powder or liquid concentrate form.

The pharmaceutical composition (or formulation) may be packaged in a variety of ways. Generally, an article for distribution includes a container that contains the pharmaceutical composition in an appropriate form. Suitable containers are well known to those skilled in the art and include materials such as bottles (plastic and glass), sachets, foil blister packs, and the like. The container may also include a tamper proof assemblage to prevent indiscreet access to the contents of the package. In addition, the container typically has deposited thereon a label that describes the contents of the container and any appropriate warnings or instructions.

The disclosed pharmaceutical compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. The pack or dispenser may also be accompanied with a notice associated with the container in form prescribed by a governmental agency regulating the manufacture, use, or sale of pharmaceuticals, which notice is reflective of approval by the agency of the form of the drug for human or veterinary administration. Such notice, for example, may be the labeling approved by the U.S. Food and Drug Administration for prescription drugs, or the approved product insert. Pharmaceutical compositions comprising a disclosed compound formulated in a compatible pharmaceutical carrier may also be prepared, placed in an appropriate container, and labeled for treatment of an indicated condition.

The exact dosage and frequency of administration depends on the particular disclosed compound, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compounds of the present disclosure.

### Aspects

The present disclosure can be described in accordance with the following numbered aspects, which should not be confused with the claims.

Aspect 1. A method of treating a subject with an aneurysm, a dissection in a blood vessel, or a combination thereof comprising administering to the subject an effective amount of one or more compounds of structure I or the pharmaceutically acceptable salt thereof:

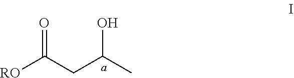

wherein R is hydrogen, an alkyl group or a hydroxyalkyl group, and the stereochemistry at carbon a is substantially R, substantially S, or racemic, or wherein the compound is a dimer or trimer of the compound of structure I.

Aspect 2. A method for reducing or preventing the risk of the formation of an aneurysm, a dissection in a blood vessel, or a combination thereof in a subject comprising administering to the subject an effective amount of one or more compounds of structure I or the pharmaceutically acceptable salt thereof:

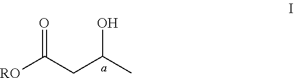

wherein R is hydrogen, an alkyl group or a hydroxyalkyl group, and the stereochemistry at carbon a is substantially R, substantially S, or racemic, or wherein the compound is a dimer or trimer of the compound of structure I.

Aspect 3. The method of Aspect 1 or 2, wherein R is hydrogen.

Aspect 4. The method of Aspect 1 or 2, wherein R is hydrogen and the stereochemistry at carbon a is substantially S.

Aspect 5. The method of Aspect 1 or 2, wherein R is a C₁ to C₁₀ hydroxyalkyl group.

Aspect 6. The method of Aspect 1 or 2, wherein R has the structure II

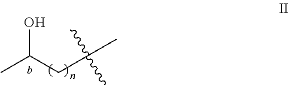

wherein n is an integer from 1 to 5 and the stereochemistry at carbon b is substantially R, substantially S, or racemic.

Aspect 7. The method of Aspect 6, wherein n is 2 and the stereochemistry at carbon b is substantially R.

Aspect 8. The method of Aspect 6, wherein n is 2 and the stereochemistry at carbon b is substantially S.

Aspect 9. The method of Aspect 1 or 2, wherein the dimer is the reaction product between a diol and the compound of structure I.

Aspect 10. The method of Aspect 9, wherein the diol comprises a C₂ to C₆ diol.

Aspect 11. The method of Aspect 9, wherein the diol comprises ethylene glycol or propylene glycol.

Aspect 12. The method of Aspect 1 or 2, wherein the trimer is the reaction product between triol and the compound of structure I.

Aspect 13. The method of Aspect 12, wherein the triol comprises a C₂ to C₆ triol.

Aspect 14. The method of Aspect 12, wherein the triol comprises glycerol.

Aspect 15. The method of Aspect 1 or 2, wherein the compound is (R)-β-hydroxybutyrate, (S)-β-hydroxybutyrate, (R)-3-hydroxybutyl-(R)-3-hydroxybutanoate, (R)-3-hydroxybutyl-(S)-3-hydroxybutanoate, (S)-3-hydroxybutyl-(R)-3-hydroxybutanoate, (S)-3-hydroxybutyl-(S)-3-hydroxybutanoate, or the pharmaceutically acceptable salt thereof.

Aspect 16. The method of Aspect 1 or 2, wherein the compound has structure Ill or the pharmaceutically acceptable salt thereof

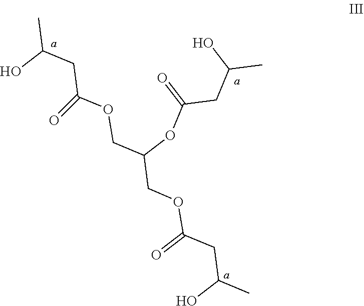 [SMILES: CC(O)CC(=O)OCC(COC(=O)CC(C)O)OC(=O)CC(C)O]

wherein the stereochemistry at carbon a is substantially S or racemic.

Aspect 17. The method in any one of Aspects 1-16, wherein the aneurysm is an abdominal aortic aneurysm, a thoracic aortic aneurysm, or a cerebral aneurysm.

Aspect 18. The method in any one of Aspects 1-16, wherein the dissection in the blood vessel is an abdominal aortic dissection or a thoracic aortic dissection.

Aspect 19. The method in any one of Aspects 1-16, wherein the compound further treats or prevents atherosclerosis.

Aspect 20. The method in any one of Aspects 1-16, wherein the compound reduces the size of aneurysmal plaque in the subject.

Aspect 21. The method in any one of Aspects 1-16, wherein the compound reduces the inner diameter, exterior diameter, or a combination thereof of the aneurysm in the subject.

Aspect 22. The method in any one of Aspects 1-16, wherein the compound recovers the stiffness of the vessel wall of the aneurysm.

Aspect 23. The method in any one of Aspects 1-16, wherein the compound converts vascular smooth muscle cells into myofibroblasts.

Aspect 24. The method in any one of Aspects 1-16, wherein the compound removes senescent cells from the vessel of the subject.

Aspect 25. The method in any one of Aspects 1-16, wherein the compound prevents the formation of senescent cells in a vessel.

Aspect 26. The method in any one of Aspects 1-16, wherein the compound converts senescent cells to non-senescent cells.

Aspect 27. The method in any one of Aspects 1-26, wherein the compound is administered as a pharmaceutical composition.

Aspect 28. The method in any one of Aspects 1-27, wherein the compound is administered orally to the subject.

Aspect 29. The method of Aspect 28, wherein the compound is administered as a capsule, a tablet, a chewing gum, a lozenge, a powder, or a beverage.

Aspect 30. The method in any one of Aspects 1-27, wherein the compound is administered intravenously, intramuscularly, subcutaneously, or intra-articularly to the subject.

Aspect 31. The method in any one of Aspects 1-27, wherein the compound is administered to the subject by a stent.

Aspect 32. The method in any one of Aspects 1-31, wherein the subject is furthered administered an effective amount of a KYNU inhibitor, a KMO inhibitor, a 3-HAO upregulator, or acipimox, or a pharmaceutically acceptable salt thereof.

Aspect 33. The method in any one of Aspects 1-32, wherein the subject is further undergoing treatment with a ketogenic diet, a ketone-ester, or a combination thereof.

Aspect 34. The method in any one of Aspects 1-33, wherein the subject has an underlying medical disorder comprising Marfan syndrome, Loeys-Dietz syndrome, aneurysms-osteoarthritis syndrome, Ehlers-Danlos syndrome, familial thoracic aortic aneurysm/dissection, Shprintzen-Goldberg syndrome, cutis laxa syndrome, aortic valve disease, arterial tortuosity syndrome, X-linked Alport syndrome, Turner syndrome, or a congenital heart malformation.

Aspect 35. The method in any one of Aspects 1-33, wherein the compound treats or prevents an aneurysm or dissection in a subject having a bicuspid aortic valve (BAV).

Aspect 36. The method in any one of Aspects 1-35, wherein the subject is identified as at risk of having an aneurysm or dissection of a blood vessel.

Aspect 37. The method of Aspect 36, wherein the subject is screened for a genetic mutation that predisposes the subject to an aneurysm, a dissection in a blood vessel, or a combination thereof

Aspect 38. The method of Aspect 37, wherein the subject has a mutated gene that encodes proteins involved in vascular smooth muscle cell contraction and adhesion to the extracellular matrix (ECM), transforming growth factor)-β signaling pathway, or smooth muscle cell metabolism.

Aspect 39. The method of Aspect 37, wherein the subject has a mutated gene, wherein the gene comprises FBN1, lysyl oxidase (LOX), smooth muscle myosin heavy chain 11 (MYH11), smooth muscle α-actin 2 (ACTA2), myosin light chain kinase (MYLK), protein kinase cGMP-dependent type 1 (PRKG1), α-1 procollagen, type III (COL3A1), TGF-β receptor type II (TGFBR2), TGF-β receptor type I (TGFBR1), TGF-β2 (TGFB2), mothers against decapentaplegic drosophila homolog 3 (SMAD3), α-1 procollagen, type I (COL1A1), α-2 procollagen, type I (COL1A2), mediator complex subunit 12 (MED12), mothers against decapentaplegic drosophila homolog 4 (SMAD4), procollagen-lysine, 2-oxoglutarate 5-dioxygenase 3 (PLOD3), endoglin (ENG), activin A receptor like Type 1 (ACVRL1), neurofibromatosis type 1 (NF1), or any combination thereof.

Aspect 40. The method in any one of Aspects 1-36, wherein the subject is screened for an elevated level of 3-hydroxyanthranilic acid (3-HAA).

Aspect 41. The method of Aspect 40, wherein the method comprises
(a) determining the level of 3-hydroxyanthranilic acid (3-HAA) present in a sample from the subject;
(b) comparing the subject's level of 3-HAA to a range of standardized levels of 3-HAA derived from individuals without an aneurysm or dissection in a blood vessel (“normal range”); and
(c) administering the compound to subject if the subject's levels of 3-HAA is greater than the normal range.

Aspect 42. A method of treating or preventing atherosclerosis in a subject comprising administering to the subject an effective amount of one or more compounds of structure I or the pharmaceutically acceptable salt thereof:

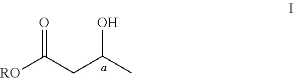

wherein R is hydrogen, an alkyl group or a hydroxyalkyl group, and the stereochemistry at carbon a is substantially R, substantially S, or racemic, or wherein the compound is a dimer or trimer of the compound of structure I.

Aspect 43. A method of converting vascular smooth muscle cells into myofibroblasts in a subject comprising administering to the subject an effective amount of one or more compounds of structure I or the pharmaceutically acceptable salt thereof:

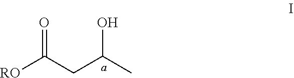

wherein R is hydrogen, an alkyl group or a hydroxyalkyl group, and the stereochemistry at carbon a is substantially R, substantially S, or racemic, or wherein the compound is a dimer or trimer of the compound of structure I.

### Examples

The following examples are put forth so as to provide those of ordinary skill in the art with a complete disclosure and description of how the compounds, compositions, articles, devices and/or methods claimed herein are made and evaluated and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure.

### Results

### Role of Vascular Senescence in AAA Development

To determine if senescence is a major cause of aneurysm development, the association between senescence and aneurysms using the angiotensin II (AII)-induced AAA model was investigated. Mice were infused AII for four weeks using osmotic mini-pump implantation into Apoe^{-/-} transgenic mouse. AII infusion significantly developed AAA (60% of incidence) accompanied by senescent cell accumulation.

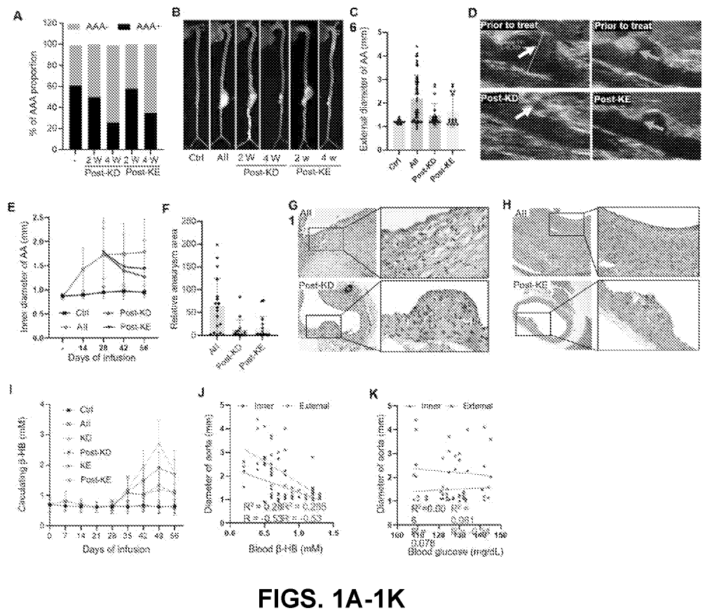

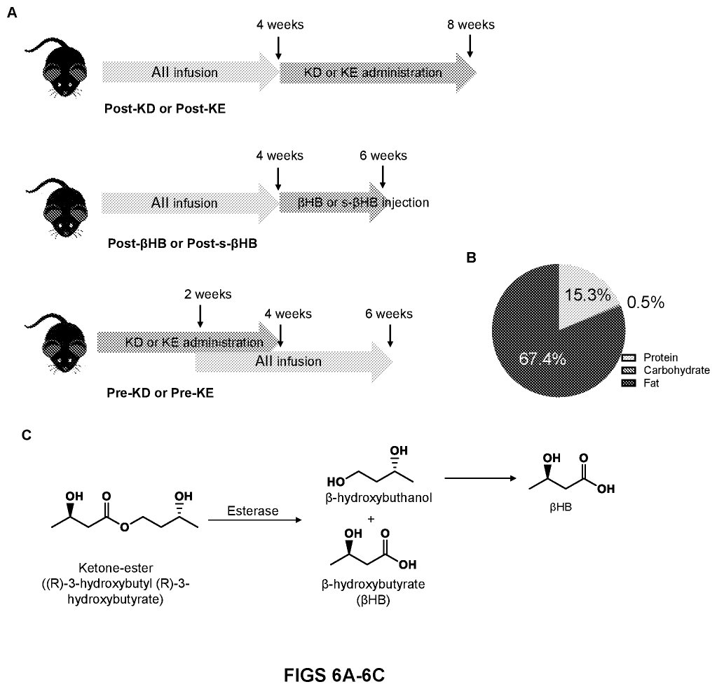

To investigate the therapeutic potential of ketotherapy for abdominal aortic aneurysm (AAA), Ketogenic-diet (KD) and Ketone-ester (KE) were provided to angiotensin II (AII)-induced mouse model of AAA (FIG. 6). AII infusion for four weeks developed AAA with 60% of incidence in C57Bl6 ApoE^{-/-} mice. After completing AII infusion, the mice were post-treated with KD and KE for two to four weeks. KD and KE both successfully reduced the proportion of AII-induced AAA. The recovered proportion after KD treatment is 35% and KE treatment is 26% (FIG. 1A), where recovered portion is meant that the mouse aortic diameter was restored to close to or the same diameter as it had prior to AAA induction. Ketotherapy specifically reduced the size of AII-induced aneurysm without morphological alterations and a blood clot in the abdominal aorta (FIG. 1B). The enlarged external diameter of AAA is decreased significantly by post-treatment of KD and KE (FIG. 1C).

To investigate the time-dependent reduction of aorta diameter, the inner diameter of the aorta using ultrasound analysis was measured. Comparing ultrasound images of identical mouse aorta proved that KD and KE treatment reverts aneurysm formation (FIG. 1D). Ketotherapy administered for two weeks significantly reduced inner diameter size. Although the efficiency of the therapeutic effect was mild after two weeks, it continues to increase up to four weeks (FIG. 1E). Ketotherapy also dramatically reduced the aneurysm area (FIG. 1F). There were no specific abnormal signs or changes in the vascular structure on the H&E staining images of the abdominal aorta. A small bulge was observed at the site of collapsed aneurysm plaque after ketotherapy (FIGS. 1G and 1H). This bulge shape is considered as neointima hyperplasia that usually takes place in the injured vessel. Since neointima hyperplasia is gradually decreased during the ketotherapy, it is believed to be a reparative process reconstructing vessel wall.

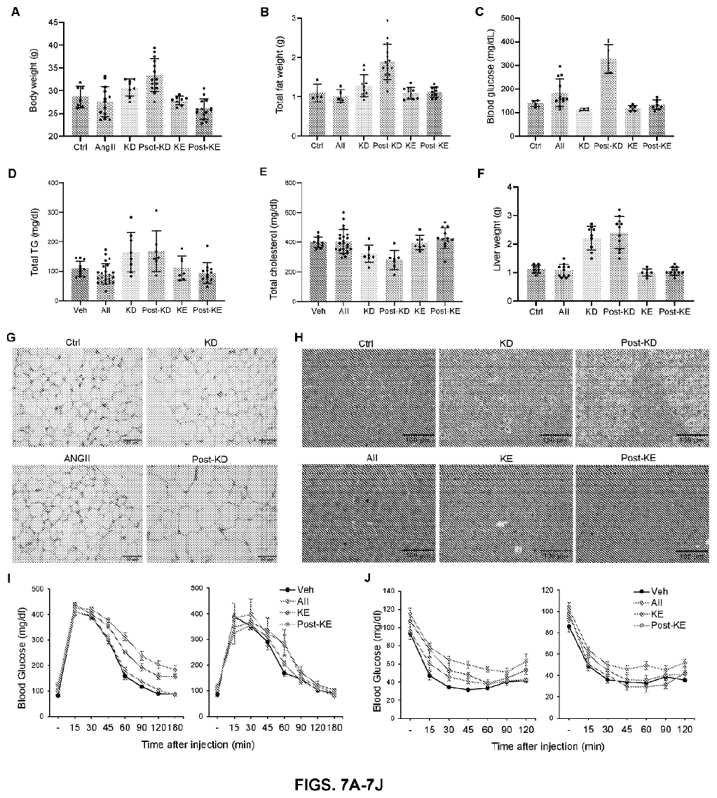

In order to identify the primary factors for reversing AAA formation, metabolic alterations by post-treatment were investigated. KD administered for four weeks causes increased body weight, fat mass, blood glucose, TG, and lipid deposition in liver which may refer as consequence of insulin resistance, whereas KE administration did not cause metabolic changes such as insulin resistance (FIG. 7). Thus, abnormal lipid metabolism caused by KD is unlikely required or helpful for AAA recovery.

Next, the negative association of circulating β-hydroxybutyrate (βHB) with severity of AAA was determined. βHB is a significant metabolite that is produced during ketotherapy; thus, circulating βHB was monitored throughout the period of AII infusion and post-treatment with ketotherapy. Post-treatment with KD (Post-KD) upregulated βHB up to 2 mM and post-treatment of KE (Post-KE) upregulated βHB up to 1.3 mM (FIG. 11). It was assumed that the different AAA recovery rates between KD and KE were due to their different circulating βHB level during treatment. Correlation analysis between the concentration of βHB and size of aneurysm diameter demonstrated that βHB is negatively associated with the size of the aneurysm (FIG. 1J) and does not correlate with glucose level (FIG. 1K). In addition, KD, which maintained a higher βHB level, showed a slightly better therapeutic effect than KE treatment. Thus, βHB is a pivotal metabolite and instrumental in reversing AAA formation.

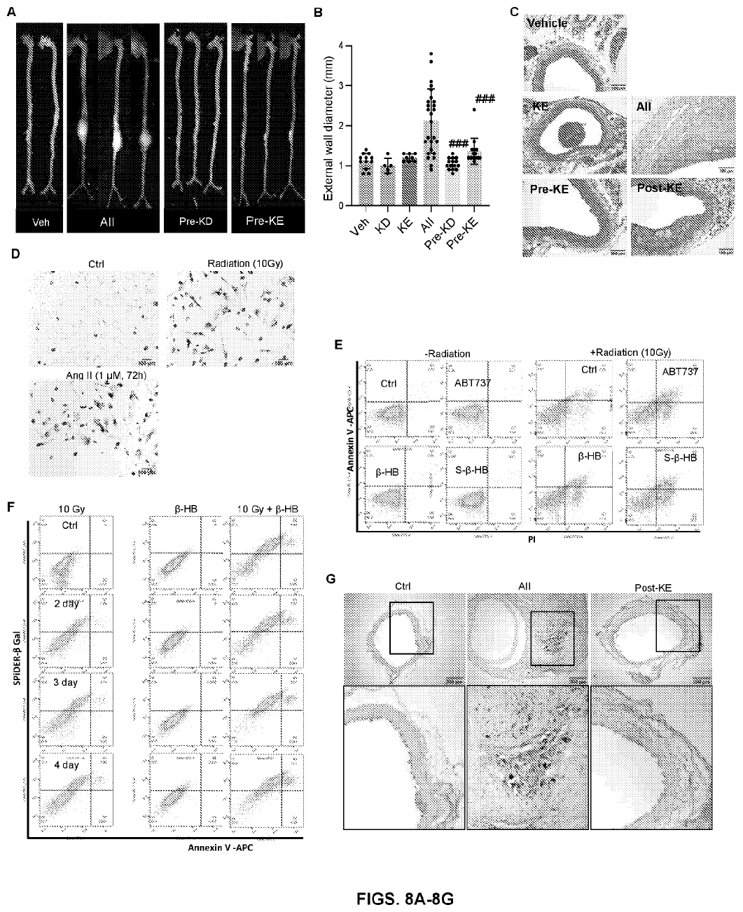

Senescence is considered to be major risk factor that promotes AAA development. It was previously demonstrated that βHB prevents vascular smooth muscle cell senescence. To determine whether vascular senescence is critical for AAA formation, senescence was prevented by pretreatment with ketotherapy. This strongly prevented AAA development (FIG. 8A-C). These results indicate that the elimination of senescent cells is essential for the recovery of AAA.

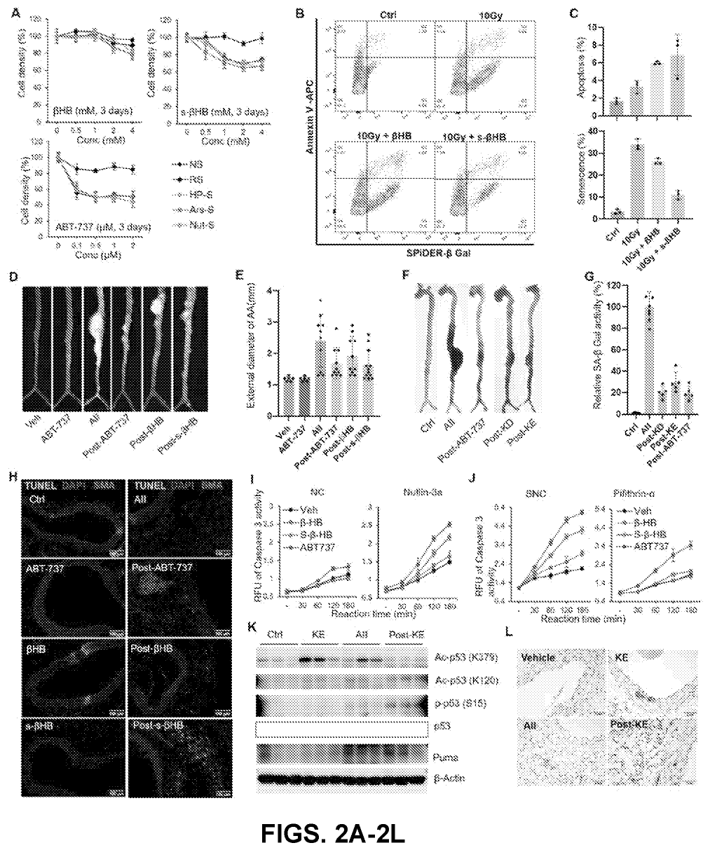

The clearance of senescent cells that accumulate during the aneurysm development is required to reduce the volume of aneurysm plaque. Thus, the senotherapeutic potential of βHB to reverse AAA progression was evaluated. A cellular viability assay using senescent SMCs was prepared. Prior to treatment, SMCs were exposed with H₂O₂ (HP), Arsenite (Ars), or Nutlin 3a (Nut) to induce premature senescence. βHB and s-βHB selectively reduced the number of senescent SMCs and not normal SMCs, which is similar to the action of ABT-737 (FIG. 2A).

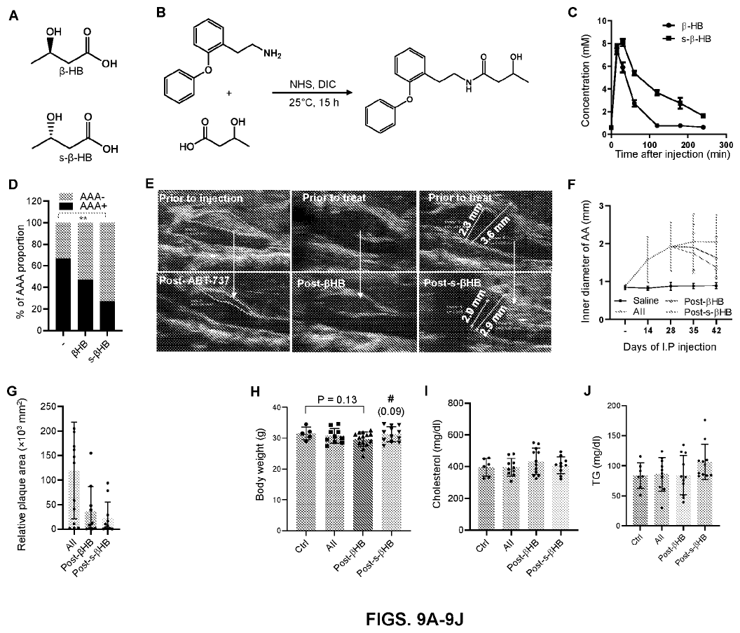

Apoptosis analysis via cytometry was conducted to clarify whether βHB selectively induced apoptosis in senescent SMCs. The non-apoptotic premature senescent SMC population, probed by SPIDER β-gal indicator, is markedly decreased, but apoptotic senescent SMCs are increased simultaneously (FIG. 2B). The result of the increasing apoptotic population of senescent cells and the reduced number of total senescent cells reveals that βHB induced targeted apoptosis of senescent SMCs (FIGS. 2C and 8D-G). Since the metabolic elimination rate of R-βHB is faster than s-βHB, an optical isomer of βHB, the potential of s-βHB as a senolytic agent is more remarkable than βHB (FIG. 9A-G) without metabolic impact (FIGS. 9H-J).

To validate whether senolytic agents can reverse aneurysm formation, ABT-737 was injected every other day for two weeks and compared to βHB or s-βHB injection. Whole aorta images showed that AAA was successfully rescued by βHB injection as well as by ABT-737 (FIG. 2D). The external diameter of the abdominal aorta was also significantly reduced by each injection (FIG. 2E). β-galactosidase activity assay on the whole aorta established that ketotherapy effectively clears senescent tissue, which is highly accumulated in aneurysm plaque (FIGS. 2F and 2G). In addition, TUNEL assay also supports that ABT-737, βHB and s-βHB induced targeted apoptosis of senescent plaque in the aneurysm (FIG. 2H).

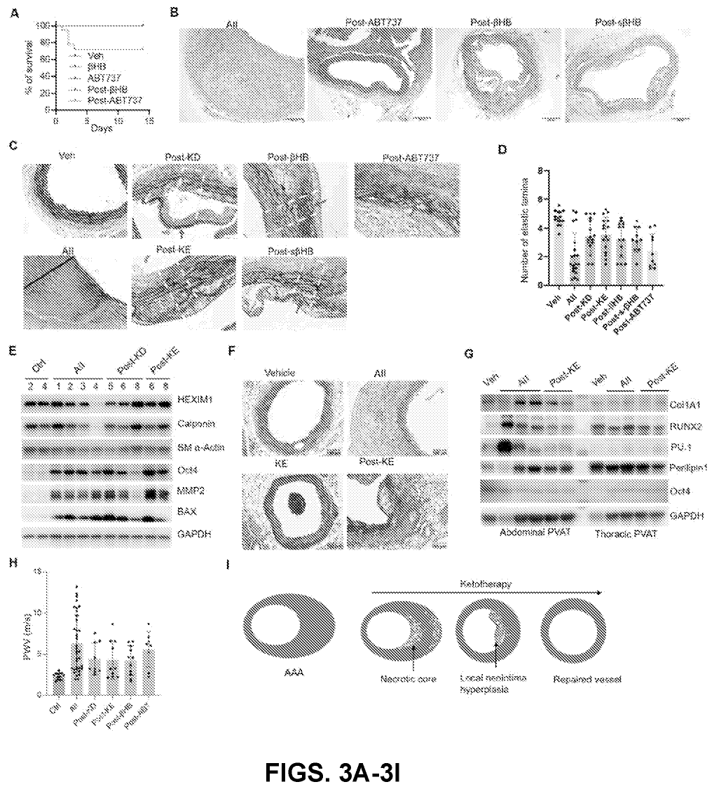

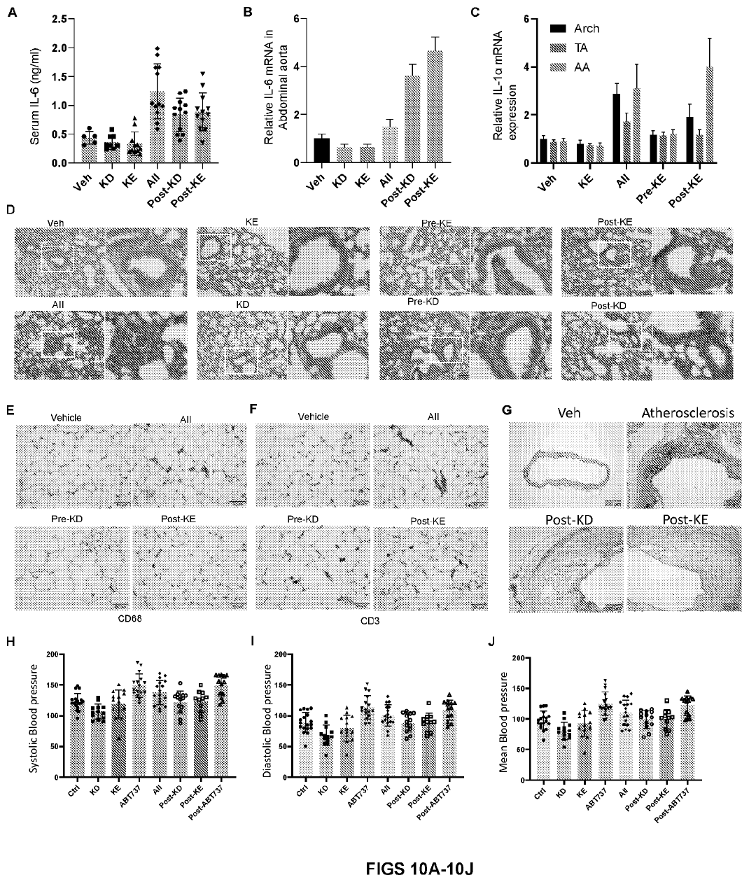

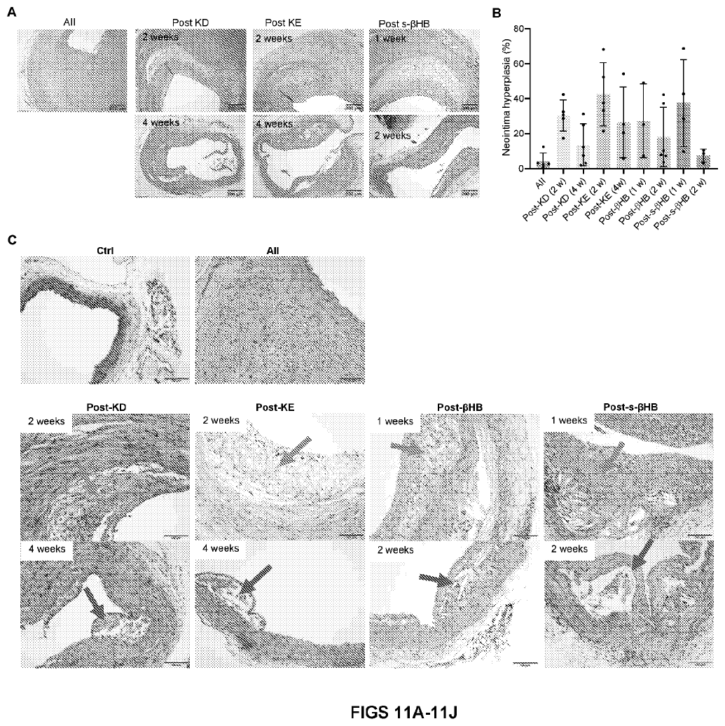

To overcome the anti-apoptotic barrier of senescence cells, senolytics usually target p53/Bcl2 cascades. Moreover, p53 is considered as a barrier to prevent apoptosis in senescence cells. Therefore, it was investigated if βHB-mediated apoptosis is p53 dependent. In normal cells (NC), ABT-737 and βHB did not induce apoptosis. However, after p53 stabilization by Nutlin 3a, ABT-737 and βHB triggers apoptosis (FIG. 2I). In the case of p53 inhibition by pifithirin-α, βHB did not induce apoptosis (FIG. 2J). To further validate p53 dependent apoptosis on AAA, p53 activation and puma expression were investigated. Post-treatment of KE activated phosphorylation of p53 and elevated puma expression, which is a transcriptional target of p53 causing apoptosis (FIGS. 2K and 3L). Together, βHB possesses a senotherapeutic potential to clear senescent plaque on AAA via regulating p53. In addition, inflammatory response accompanying the apoptosis of senescence cells was observed specifically in the abdominal aorta tissue, while systemic inflammation decreased during ketotherapy (FIGS. 10A-F). Although there was an increase of inflammation caused by apoptosis of senescent cells, ketotherapy did not develop atherosclerosis, which is known as an inflammatory disease (FIG. 10G). Interestingly, the senotherapeutic effect of βHB is consistent with ABT-737; however, βHB treatment exhibited different blood pressure changes after treatment. Ketotherapy can lower the blood pressure caused by AII infusion, which is not the case with ABT-737 (FIGS. 10H-J).

Unexpectedly, although ABT-737 successfully reduced the volume of aneurysm plaque, 30% of the mice injected with ABT-737 had an abdominal rupture, resulting in sudden death (FIG. 3A). ABT-737 caused aortic dissection, and blood clots were detected inside of aneurysm plaque. Ketotherapy is accompanied by partial neointima hyperplasia after recovery. However, ABT-737 was associated with smaller and more minor neointima hyperplasia than ketotherapy (FIG. 3B). It was assumed that vascular regeneration occurs in neointima hyperplasia to prevent aortic dissection and improve vascular integrity. Since elastic lamina breakage is the leading cause of aortic dissection, alteration of the elastic lamina was investigated by Verhoeff Van Gieson/EVG stain. Interestingly, the broken elastic lamina in an aneurysm is changed to branched elastic fibers during ketotherapy. Compared to ketotherapy, ABT-737 treated aorta has less branched elastic fibers (FIG. 3C). Multiple ketotherapy recovers the number of elastic layers, but ABT-737 is less effective on elastic recovery (FIG. 3D).

For vascular wall regeneration, sufficiently matured SMCs are critical to improving vascular function. During the AII-induced AAA development, degeneration of SMC gradually progressed, resulting in the reduction of the SMC markers, HEXIM1, Calponin 1, and SM α-Actin. At the same time, dedifferentiation factors Oct4 and MMP2 increased throughout the AAA development. Surprisingly, post-treatment of KD and KE reversed all the markers of dedifferentiation and simultaneously recovered SMC markers (FIG. 3E). Recovery of elastic lamina and SMC layer reveals regeneration of tunica media on abdominal aorta. In addition, loss of connective tissue in AAA is recovered as well. In AAA, ECM networks are degraded, and collagen, a main component of ECM is diffused without a structural scaffold. Collagen network is reconstituted at the perivascular area after post-treatment with KE (FIG. 3F). Moreover, KE treatment also specifically reverses degenerative markers, col1A1, RUNX2, and PU.1 in abdominal perivascular adipose (PVAT), not in thoracic PVAT (FIG. 3G). Together, all the recovered tissue indicated improvement of vascular integrity. Therefore, abdominal aorta stiffness was measured to validate the recovery of vascular function of the aorta. Only ketotherapy such as KD, KE and βHB treatment ameliorated vascular stiffness caused by AAA formation (FIG. 3H). According to the previous results, ketotherapy induced the regeneration of multiple types of tissues and clearance of senescent cells to repair damaged vessels well.

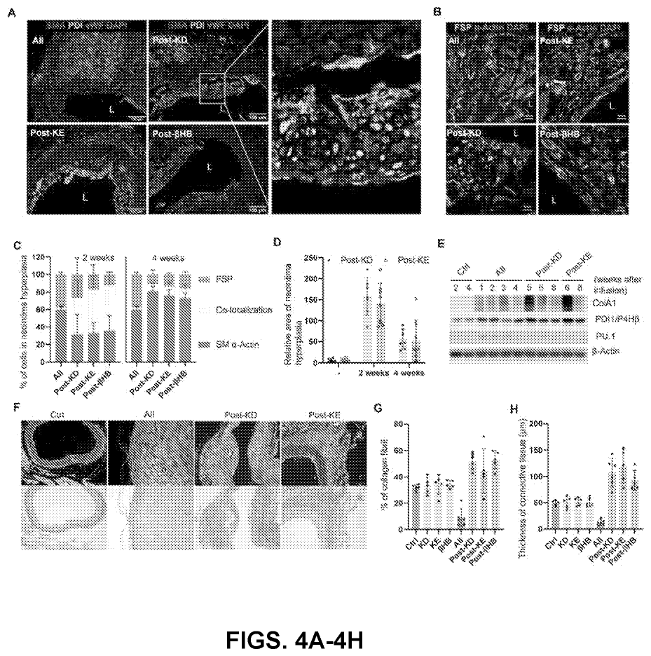

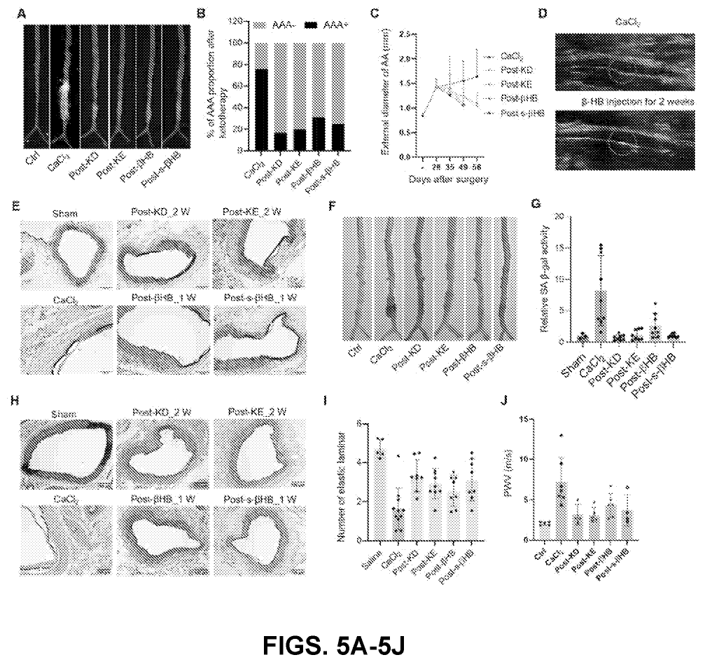

Neointima hyperplasia that was observed after ketotherapy but not observed with ABT-737 (FIG. 3I) was investigated. Fibroblasts repair injured tissues by reconstituting dynamic and diverse type of vascular tissues such as endothelial, smooth muscle, and connective tissues through regenerative strategies. It was supposed that the multifaceted regeneration by ketotherapy is likely explained by fibroblast-associated vascular remodeling. As discussed above, neointima hyperplasia, which appears after ketotherapy, is considered a source of fibroblast-mediated tissue repairment. First, the alteration of the fibroblast population in AAA tissues by comparing recovered aorta after ketotherapy was investigated. PDI positive cells, referred to as fibroblast, are mostly accumulated in neointima hyperplasia (FIG. 4A). Fibroblast and smooth muscle cells are independently positioned in the aneurysm area. However, the cells in neointima hyperplasia after ketotherapy present colocalization of fibroblast surface protein (FSP) and smooth muscle α-actin (FIG. 4B). Moreover, PDI and FSP positive cells are also clearly located at the edge of neointima hyperplasia that is considered to EC layer (FIGS. 4A and 4B).

To confirm the possibility that the therapeutic effect of βHB on AAA is not only applied to AII-induced AAA, the CaCl₂)-induced AAA animal model was also examined as well (FIG. 5A). Local stimulation by CaCl₂) resulted in a regional selective aneurysm in the abdominal aorta with 76% incidence. Effective recovery from CaCl₂)-induced AAA by KD, KE and βHB therapy validated their potential as novel therapeutic options for calcification and inflammation-mediated AAA (FIG. 5B). Even though CaCl₂)-induced AAA development persisted after four weeks, post-treatment with KD, KE and βHB inhibited AAA progression (FIG. 5C). This shows that ketotherapy is effective in preventing AAA incidence as well as reversing AAA. Indeed, pretreatment with KD, KE strongly inhibited AII-induced AAA development.

Additionally, βHB repaired abnormal curvature on aortic wall caused by CaCl₂)-induced injury (FIG. 5D). CaCl₂) treatment give rise to enlarged abdominal aorta diameter along with loss of tunica media. Structural improvement of CaCl₂)-induced AAA after ketotherapy was validated by H&E staining (FIG. 5E). Next, a SA β-gal assay was performed to determine that CaCl₂)-induced AAA is also associated with senescence. Consistent with previous results, βHB significantly reduced abdominal specific senescent cells that were derived by local CaCl₂) stimulation (FIGS. 5F and 5G). Additionally, investigation of the elastic laminar confirmed that βHB improved vascular integrity reduced by CaCl₂)-induced AAA formation (FIGS. 5H and 5I). As a result of the clearance of senescent cells and restoration of the elastin laminar, ketotherapy improved aortic stiffness caused by CaCl₂)-induced AAA formation (FIG. 5J).

### Improved Therapeutic Efficacy and Much Longer Half-Life of S-βHB, an Optical Isomer of βHB Over (R)-BHB

Since βHB is used as an energy source in various organs including the heart and muscles, the metabolic process very rapidly consume βHB resulting in a shortage of βHB in physiology. This reduced intracellular retention time becomes an obstacle that hinders sufficient action as a drug. Therefore, it is important to improve therapeutic effects of βHB by improving pharmacokinetic parameters related to drug metabolism. BDH1, a significant enzyme that converts βHB as a resource for acetyl-coenzyme A (Ac-COA), selectively converts D-βHB (R-βHB, or βHB) to acetylacetonates (AcAc). Using the optical isomer of βHB, L-βHB (s-βHB), allows processing independent of the metabolic pathway controlled by BDH1. Therefore, s-βHB possesses a relatively longer half-life that is more advantageous in terms of drug metabolism than that of βHB.

We established an in vivo tolerance test of βHB and s-βHB (FIGS. 9A and 9B). This test determined that the half-life of βHB is 50 min and s-βHB is 120 min (FIG. 9C). As we expected, s-βHB treatment showed significant improvement in the recovery rate of aneurysms compared to the βHB treatment. FIG. 9D showed that the incidence of AAA by angiotensin II infusion is 67%, and post-treating s-βHB dramatically reduced the AAA rate to 27%, while βHB reduced it to 47%. It showed that s-βHB reveals significant improvement in the number of mice that recovered from AAA (Chi-square test, ^{####}P<0.0001, n=10). The reduction of the inner diameter of the aortic wall is also dramatic in the treatment of s-βHB in FIG. 9F (Two-way ANOVA, ^{###}P<0.005, n=10). Moreover, s-βHB reduced lesion area relatively more effective than βHB (FIG. 9G). However, there is no significant alteration in body weight and lipids levels (FIGS. 9H-9J) indicating that s-βHB did not affect metabolic pathways.

We considered that selective elimination of senescent vascular cells is the critical for reversal of AAA. Therefore, we supposed that βHB and s-βHB act as senolytics. To validate the potential of senolytics, we first examined the apoptosis of senescent SMCs after treating βHB and S-βHB. s-βHB significantly reduced the number of senescent SMCs similar to ABT-737, a representative senolytic, while βHB slightly reduced the senescent SMCs (FIG. 2A). To determine if reduction of senescent cells by βHB, or s-βHB is through apoptosis, we used FACS analysis applying apoptosis markers, PI and Annexin V. FACS analysis determined that s-βHB-induced selective apoptosis of senescent SMCs is more effectively than βHB (FIGS. 2B, 2C). Statistical analysis reveals significant improvement in the senotherapeutic potential of s-βHB compared to βHB (Student's T-test, ^{###}P<0.005, n=3). Along with our previous results that s-βHB is more effective than βHB in reducing aneurysms, the external diameter is also strongly reduced by s-βHB treatment (FIGS. 2D, 2E). Student's T-test for s-βHB vs βHB, P=0.08, n=12.

To further validate the therapeutic efficacy of s-βHB on aneurysms, we used a CaCl₂-induced AAA animal model as well as an angiotensin II infusion model. We consistently observed that s-βHB effectively reversed aneurysms developed by CaCl₂) stimulation (FIGS. 5A and 5B). The recovery rate of AAA by s-βHB showed similar effects to KD and KE, while βHB showed less effectiveness compared to s-βHB, KD, and KE treatment. Time-dependent reduction of the external diameter of the aortic wall after treatment reveals that s-βHB is more effective than βHB (FIG. 5C). Two-way ANOVA, s-βHB vs βHB, #P<0.01, n=10. Since the elimination of senescent SMCs is critical for the recovery of AAA, we investigated that s-βHB is also effective to remove senescent cells in CaCl₂)-induced aneurysms. SA β-gal assay on CaCl₂)-induced aneurysms showed that s-βHB significantly reduced senescent cells along with the recovery of aneurysms. However, βHB treatment is less effective than KD, KE and s-βHB. Student's T-test for s-βHB vs βHB, ^{#}P<0.05, n=8. Taken together, s-βHB is more effective than βHB in aneurysm therapy due to an advantage in retention time.

### Role of Oct4 in AAA

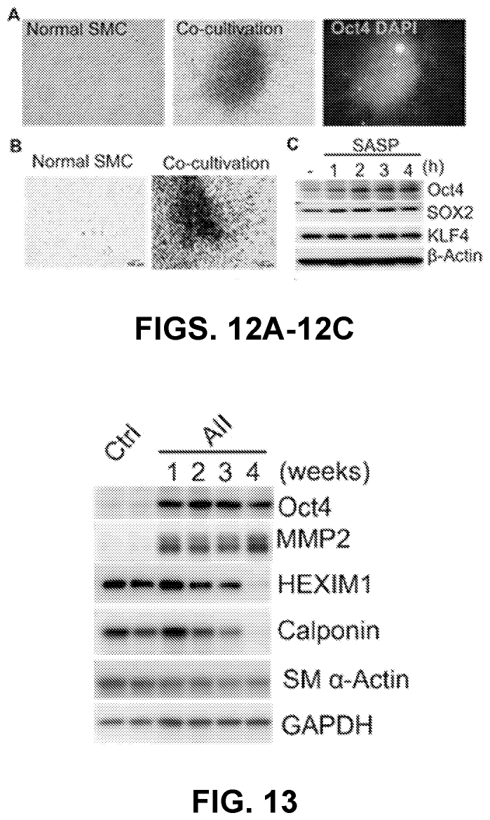

A localized senescent cell clump was identified in co-culture conditions similar to the accumulation of severe senescence in aneurysms (FIG. 8A). Long-term cultivation of SMCs with senescent cells forms a multilayered cell mass (FIG. 12A). Usually, stem-cell-related gene activation enables the formation of the spheroid. Thus, we firstly determine the expression of Oct4, a crucial stem cell marker, in the clump of senescent SMCs. Oct4 level is significantly and specifically increased in the clump of senescent SMCs compared to proximal cells (FIG. 12A). Interestingly, spheroid formation of SMCs caused severe senescence similar to its accumulation in aneurysms. Thus, we expect SASP-induced Oct4 elevation does not possess the reprogramming function. Imperfect reprogramming is determined by other reprogramming factors (Sox2 and Klf4) that do not activate during SASP stimulation (FIG. 12C). These results provide a novel hypothesis that SASP, secreted from senescent cells, activates Oct4 in normal SMCs that may initiate dedifferentiation of SMCs, which are failed to cellular reprogramming. Oct4-induced dedifferentiation overcomes contact inhibition to form a rapid increase of senescent cell clump.

Loss of contractile phenotype of SMCs promotes cell proliferation and migration. Additionally, increased matrix metalloproteinases (MMP2) regulate the outgrowth of cells. Thus, we considered that imperfect reprogramming by Oct4 induced dedifferentiation of SMCs to form rapid growth of asymmetric aneurysms. Our in vivo results also support the hypothesis that SASP-induced Oct4 has a limitation of reprogramming, thereby it induces dedifferentiation of SMCs. Oct4 and MMP2 are stably upregulated in aorta tissues after AII infusion, while SMC markers (HEXIM1, Calponin, and SM α-Actin) gradually decrease. (FIG. 13).

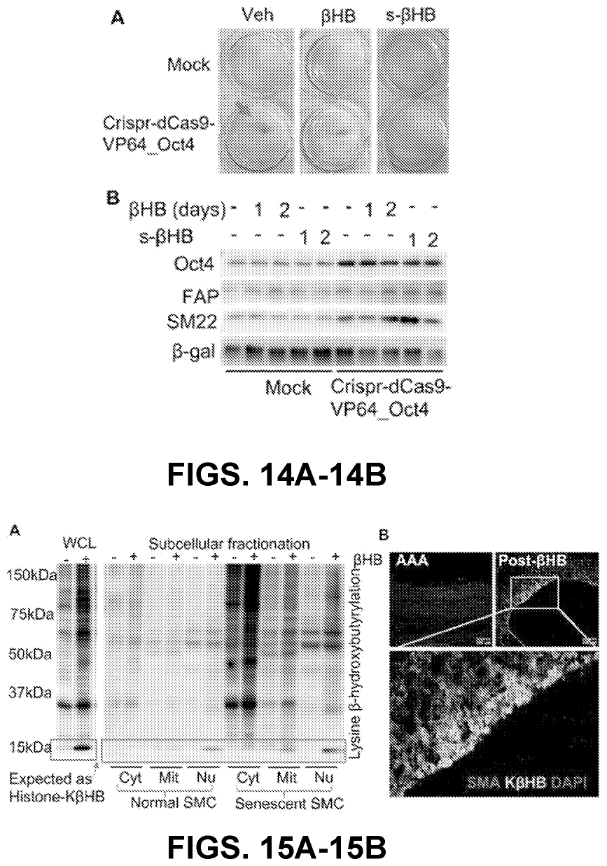

We proposed that Oct4 upregulation in senescence forms senescent spheroid-like cell mass and asymmetric configuration of aneurysms. To validate our central hypothesis, we overexpressed Oct4 in senescent SMCs using the CRISPR-dCas9-VP64 system to determine the role of Oct4. Surprisingly, overexpression of Oct4 in senescent SMCs dramatically increased localized senescent cell clumps, while βHB treated SMCs reduced senescent cells. Localized increase of senescence may be highly associated with asymmetric dilation of aneurysms. Additionally, βHB and s-βHB treatment significantly reverses or prevents the development of localized senescence (FIG. 14A). We supposed that βHB prevents or reverses Oct4-induced senescence of SMCs, consequently reducing the size of aneurysms. To investigate the role of βHB in aneurysms, we assess the possibility of cellular reprogramming in senescent SMCs in the condition of Oct4 overexpression. Interestingly, βHB does not recover SM22, a smooth muscle cell marker in senescent SMCs without Oct4 activation, while βHB recovers SMC maturation marker (SM22) and reduced senescence marker (β-galactosidase) in Oct4 overexpressed-senescent cells (FIG. 14B). In particular, a moderate increase of FAP (Fibroblast Activation Protein) provides a potential for reprogramming resulting in transdifferentiation of SMCs to myofibroblast. Recovered SM22 expression in Oct4 overexpressed senescent SMCs supposed that βHB converts the Oct4 function in dedifferentiation into a reprogramming-related role. We considered an βHB-induced epigenetic alteration of senescent cells contributing to Oct4-dependent cellular reprogramming based on this unique observation.

Since epigenetic regulation directly affects the activity of transcriptional factors, such as Oct4, we evaluated if βHB is related to histone remodeling. βHB is a substrate of acyltransferase resulting in β-hydroxybutyrylation, a unique posttranslational modification that controls protein activity or gene expression. We hypothesize that β-hydroxybutyrylation on histone induces histone remodeling facilitating cellular reprogramming. To determine that β-hydroxybutyrylation (KβHB) is observed in senescent SMCs or aneurysms, we applied a specific antibody detecting KβHB modified protein. Western blot analysis is carried out using KβHB specific antibody (PTM Biolabs, Cat #: PTM-1201) to validate that treating βHB increases KβHB modified proteins throughout the diverse range of molecular weight. Notably, p53 and histones are identified to be modified with KβHB.³⁶⁻³⁸ Hence, we expect the increased immunoblot signal around 15 kDa of protein size may be KβHB modified histones (FIG. 15A). The protein presumed to be histone-KβHB is mainly present in the nucleus fraction and is upregulated significantly in senescent SMCs (FIG. 15A). In vivo results also coincided with in vitro results that treating βHB increases KβHB modified protein in nuclear, considered as β-hydroxybutyrylation of histone (FIG. 15B). Together, it is supposed that β-hydroxybutyryl histone triggers the reprogramming process of senescent SMCs by regulating Oct4, which is overexpressed in aneurysms.

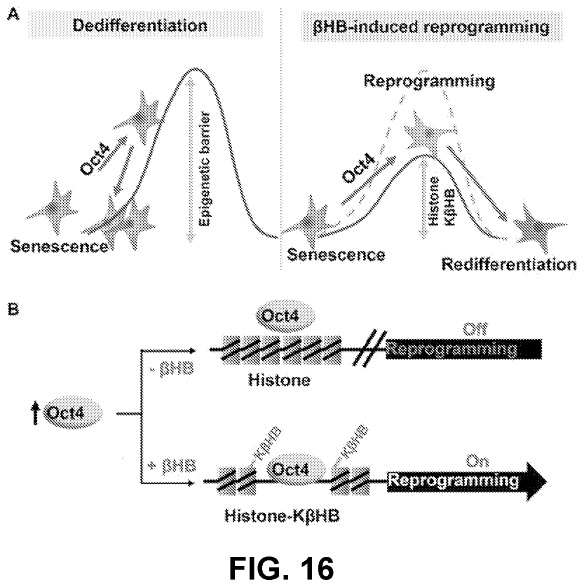

Partial reprogramming is a unique cellular process remodeling the aged tissue to ameliorate age-associated hallmarks. Partial reprogramming stimulated by OSKM (Oct4, Sox2, Klf4, and c-Myc) improves tissue homeostasis, resulting in prolonged lifespan in a mouse model of premature aging. We expect that βHB may facilitate the Oct4-mediated reprogramming process by epigenetic regulation. Somatic cells suppress reprogramming due to an obstacle for the cell, called the “epigenetic barrier,” thus preventing unwanted gene expression from other lineages. Despite the increase in expression of Oct4 in the aneurysms, cellular reprogramming is limited without overcoming the epigenetic barrier. Our central hypothesis is that βHB lowers the epigenetic barrier to transform senescent cells into redifferentiated cells through reprogramming (FIG. 16A). Chromatin remodeling regulated by histone modification is highly implicated in epigenetics. Histone modifications such as methylation/acetylation can alter chromatin structure, resulting in transcriptional activation.

Transformation of Senescent SMCs into Mature SMCs and Myofibroblasts

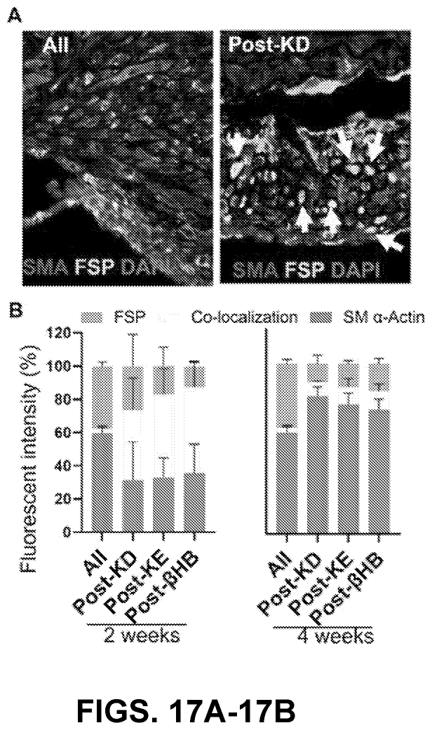

SMCs and fibroblasts possess remarkable phenotypic plasticity that allows adaptation by repairing injured tissue. Moreover, histone modification programs specify the SMC differentiation and regulate SMC plasticity. Fibroblast reveals similar characteristics with SMCs in contractility-related gene expression, including SM α-actin and myosin. Fibroblasts are critical in supporting routine wound healing, involved in essential processes such as breaking down the fibrin clot, creating new extracellular matrix (ECM) and collagen structures to support the other cells associated with effective wound healing, and contracting the wound. Myofibroblast plays a significant role in the contractility of the aortic wall and mainly forms perivascular connective tissues to prevent dilation of the aortic wall. Newly generated myofibroblast is observed in the aortic wall post-treated with KD, while resident SMCs and fibroblasts are positioned independently in aneurysms. (FIG. 17A). SM α-Actin and fibroblast activation protein (FSP) co-staining, representing myofibroblast, increases by post-treatment of KD, KE, and βHB (FIG. 17B). Validating cell fate shift of SMC into myofibroblasts will support the Oct4-induce reprogramming of senescent SMCs under the histone remodeling by β-hydroxybutyryl histone.

Recovery of matured SMCs by reprogramming is critical for tunica media reconstruction, which prevents ruptured aneurysms. During the AII-induced AAA development, degeneration of SMC gradually progressed, resulting in the reduction of the SMC markers, HEXIM1, Calponin 1, and SM α-Actin. At the same time, dedifferentiation factors, Oct4 and MMP2, are increased throughout the AAA development (FIG. 3E). Surprisingly, ketotherapy (KD and KE) reversed all the dedifferentiation markers and simultaneously recovered SMC markers (FIG. 3E). It also supports that ketotheapy induces SMC redifferentiation by partial reprogramming of senescent SMCs.

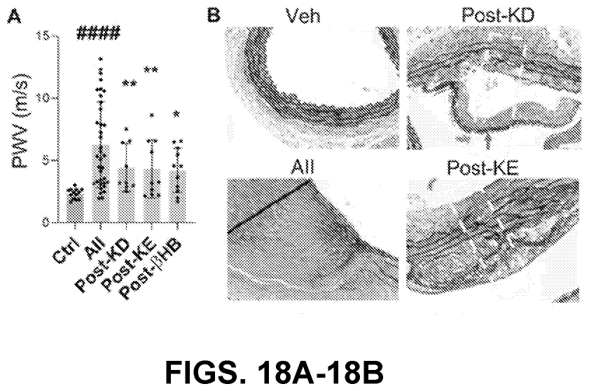

We measured Pulse Wave Velocity (PWV) in AAA mice to assess the aorta stiffness caused by aneurysms. Ketotherapy (KD, KE, and βHB) reversed aneurysm-associated aortic stiffness that may be a consequence of partial reprogramming of senescent cells (FIG. 18A). During AAA development, ECM networks, elastic fibers, and collagen are degraded and diffused without a structural scaffold resulting in loss of contractility of the arterial wall. Additionally, ketotherapy recovers elastic lamina in the SMC layer that reveals regeneration of tunica media on the abdominal aorta (FIG. 18B). Together, all the therapeutic outcomes present an improvement in vascular homeostasis.

### Ketone Ester (KE) Administration Reduces the Incidence of Both Thoracic Aortic Dissection (TAD) and Abdominal Aortic Dissection in Mouse Model of Aortic Dissection In Vivo.

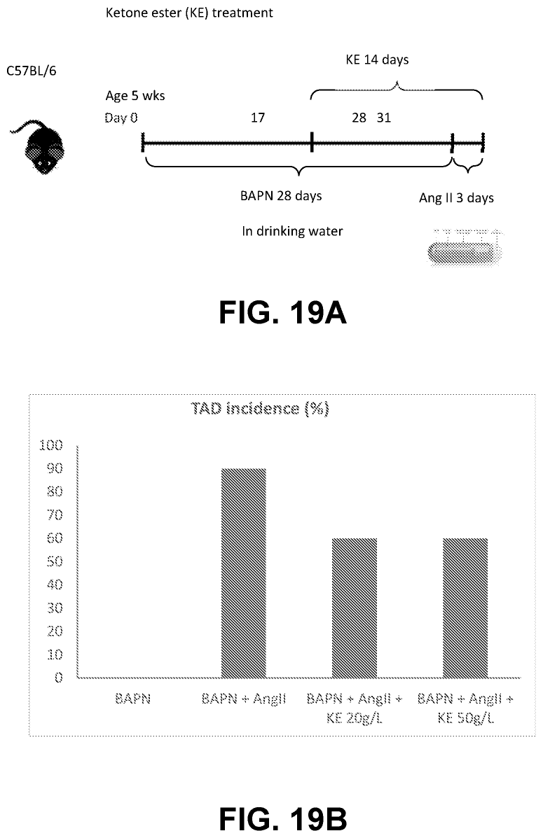

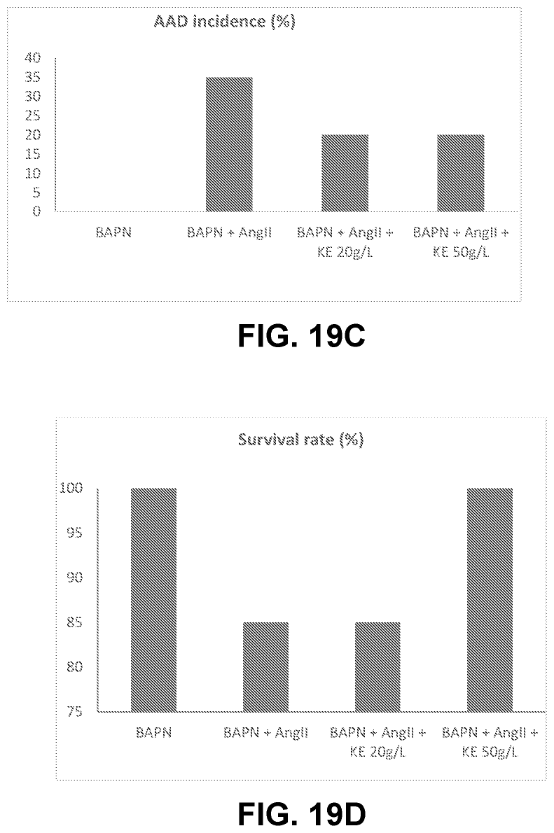

FIG. 19A shows the schematic description of aortic dissection model and ketone treatment. Five week old C57BL/6 mice were continuously treated with β-aminopropionitrile (BAPN, 3 g/L/day in drinking water)) for 28 days. To test the impacts of ketone body, BAPN-treated mice at day 17 to day 31 were given ketone ester (KE, D-BHB 1-3 butanediol monoester) (20 g/L and 50 g/L) in drinking water. Further, the BAPN-treated mice, with or without ketone treatment, were continuously infused with angiotensin (AngII, 1.44 mg/kg/day) for three days via subcutaneously implanted osmotic pumps). Three days after AngII infusion, the mice were euthanized for assay aortic dissection. FIG. 19B shows that KE significantly reduces the incidence of TAD. FIG. 19C shows that KE significantly reduces the incidence of AAD. FIG. 19D shows that high dose of KE (50 g/L) significantly increase the survival rate. #p<0.05, BAPN verse BAPN+AngII; †p<0.05 BAPN+AngII verse BAPN+AngII+KE; BAPN (n=10); BAPN+AngII (n=20); BAPN+AngII+KE 50 g/L (n=20); BAPN+AngII+KE 50 g/L (10).

### SUMMARY

βHB provides a viable approach for reversing or preventing AAA, an age-associated vascular disease with limited pharmacological intervention.²⁴ Senescent SMCs accumulated during AAA development are essential units to restore the injured aorta during AAA development. βHB removes senescence SMCs that cause degenerative plaque formation of an aneurysm. Simultaneously, βHB repairs the injured aorta via trans-differentiation of fibroblast to EC, SMC which consist in the aorta wall. Fibroblasts reconstruct connective tissue to restore vessel tone, causing shrink of the dilated vessel wall. Fibroblasts safely repair the injured aorta without dissection and rupture.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

### REFERENCES

1 Lee, K. M. et al. Effects of anatomical characteristics as factors in abdominal aortic aneurysm rupture: CT aortography analysis. Medicine (Baltimore) 96, e7236, doi:10.1097/MD.0000000000007236 (2017).
2 Singh, K., Bonaa, K. H., Jacobsen, B. K., Bjork, L. & Solberg, S. Prevalence of and risk factors for abdominal aortic aneurysms in a population-based study: The Tromso Study. Am J Epidemiol 154, 236-244 (2001).
3 Howard, D. P. et al. Age-specific incidence, risk factors and outcome of acute abdominal aortic aneurysms in a defined population. Br J Surg 102, 907-915, doi:10.1002/bjs.9838 (2015).
4 Umebayashi, R., Uchida, H. A. & Wada, J. Abdominal aortic aneurysm in aged population. Aging (Albany NY) 10, 3650-3651, doi:10.18632/aging. 101702 (2018).
5 Lindeman, J. H. & Matsumura, J. S. Pharmacologic Management of Aneurysms. Circ Res 124, 631-646, doi:10.1161/CIRCRESAHA. 118.312439 (2019).
6 Gadowski, G. R., Pilcher, D. B. & Ricci, M. A. Abdominal aortic aneurysm expansion rate: effect of size and beta-adrenergic blockade. J Vasc Surg 19, 727-731, doi:10.1016/s0741-5214 (94) 70048-6 (1994).
7 Assar, A. N. & Zarins, C. K. Ruptured abdominal aortic aneurysm: a surgical emergency with many clinical Postgrad Med presentations. 85, 268-273, J doi:10.1136/pgmj.2008.074666 (2009).
8 Bandera-Merchan, B., Boughanem, H., Crujeiras, A. B., Macias-Gonzalez, M. & Tinahones, F. J. Ketotherapy as an epigenetic modifier in cancer. Rev Endocr Metab Disord, doi:10.1007/s11154-020-09567-4 (2020).
9 Scholl-Burgi, S. et al. Ketogenic diets in patients with inherited metabolic disorders. J Inherit Metab Dis 38, 765-773, doi:10.1007/s10545-015-9872-2 (2015).
10 Wlodarek, D. Role of Ketogenic Diets in Neurodegenerative Diseases (Alzheimer's Disease and Parkinson's Disease). Nutrients 11, doi:10.3390/nu11010169 (2019).
11 Liao, S., Curci, J. A., Kelley, B. J., Sicard, G. A. & Thompson, R. W. Accelerated replicative senescence of medial smooth muscle cells derived from abdominal aortic aneurysms compared to the adjacent inferior mesenteric artery. J Surg Res 92, 85-95, doi:10.1006/jsre.2000.5878 (2000).
12 Chen, H. Z. et al. Age-Associated Sirtuin 1 Reduction in Vascular Smooth Muscle Links Vascular Senescence and Inflammation to Abdominal Aortic Aneurysm. Circ Res 119, 1076-1088, doi:10.1161/CIRCRESAHA. 116.308895 (2016).
13 He, S. & Sharpless, N. E. Senescence in Health and Disease. Cell 169, 1000-1011, doi:10.1016/j.cell.2017.05.015 (2017).
14 Collado, M. & Serrano, M. Senescence in tumours: evidence from mice and humans. Nat Rev Cancer 10, 51-57, doi:10.1038/nrc2772 (2010).
15 Kim, K. H., Chen, C. C., Monzon, R. I. & Lau, L. F. Matricellular protein CCN1 promotes regression of liver fibrosis through induction of cellular senescence in hepatic myofibroblasts. Mol Cell Biol 33, 2078-2090, doi:10.1128/MCB.00049-13 (2013).
16 Wang, J. C. & Bennett, M. Aging and atherosclerosis: mechanisms, functional consequences, and potential therapeutics for cellular senescence. Circ Res 111, 245-259, doi:10.1161/CIRCRESAHA. 111.261388 (2012).
17 Gunasekaran, U. & Gannon, M. Type 2 diabetes and the aging pancreatic beta cell. Aging (Albany NY) 3, 565-575, doi:10.18632/aging. 100350 (2011).
18 Bredesen, D. E. et al. Reversal of cognitive decline in Alzheimer's disease. Aging (Albany NY) 8, 1250-1258, doi:10.18632/aging. 100981 (2016).
19 Finkel, T., Serrano, M. & Blasco, M. A. The common biology of cancer and ageing. Nature 448, 767-774, doi:10.1038/nature05985 (2007).
20 Tominaga, K. The emerging role of senescent cells in tissue homeostasis and pathophysiology. Pathobiol Aging Age Relat Dis 5, 27743, doi:10.3402/pba.v5.27743 (2015).
21 Childs, B. G., Baker, D. J., Kirkland, J. L., Campisi, J. & van Deursen, J. M. Senescence and apoptosis: dueling or complementary cell fates? 15, 1139-1153, doi:10.15252/embr.201439245 (2014).
22 Roos, C. M. et al. Chronic senolytic treatment alleviates established vasomotor dysfunction in aged or atherosclerotic mice. Aging Cell 15, 973-977, doi:10.1111/acel.12458 (2016).
23 Rhinn, M., Ritschka, B. & Keyes, W. M. Cellular senescence in development, regeneration and disease. Development 146, doi:10.1242/dev.151837 (2019).
24 Figueira, I. et al. Interventions for age-related diseases: Shifting the paradigm. Mech Ageing Dev 160, 69-92, doi:10.1016/j.mad.2016.09.009 (2016).
25 Mao, A. S. & Mooney, D. J. Regenerative medicine: Current therapies and future directions. Proc Natl Acad Sci USA 112, 14452-14459, doi:10.1073/pnas. 1508520112 (2015).

1. A method of treating a subject with an aneurysm, a dissection in a blood vessel, or a combination thereof or reducing or preventing the risk of the formation of an aneurysm, a dissection in a blood vessel, or a combination thereof in a subject comprising administering to the subject an effective amount of one or more compounds of structure I or the pharmaceutically acceptable salt thereof: 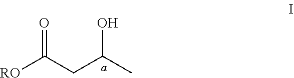
wherein R is hydrogen, an alkyl group or a hydroxyalkyl group, and the stereochemistry at carbon a is substantially R, substantially S, or racemic, or wherein the compound is a dimer or trimer of the compound of structure I.

2. (canceled)

3. The method of claim 1, wherein R is hydrogen.

4. The method of claim 1, wherein R is hydrogen and the stereochemistry at carbon a is substantially S.

5. The method of claim 1, wherein R is a C₁ to C₁₀ hydroxyalkyl group.

6. The method of claim 1, wherein R has the structure II 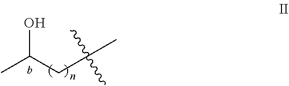
wherein n is an integer from 1 to 5 and the stereochemistry at carbon b is substantially R, substantially S, or racemic.

7. The method of claim 6, wherein n is 2 and the stereochemistry at carbon b is substantially R or substantially S.

8. (canceled)

9. The method of claim 1, wherein the dimer is the reaction product between a diol and the compound of structure I.

10. The method of claim 9, wherein the diol comprises a C₂ to C₆ diol.

11. The method of claim 9, wherein the diol comprises ethylene glycol or propylene glycol.

12. The method of claim 1, wherein the trimer is the reaction product between triol and the compound of structure I.

13. The method of claim 12, wherein the triol comprises a C₂ to C₆ triol.

14. The method of claim 12, wherein the triol comprises glycerol.

15. The method of claim 1, wherein the compound is (R)-β-hydroxybutyrate, (S)-β-hydroxybutyrate, (R)-3-hydroxybutyl-(R)-3-hydroxybutanoate, (R)-3-hydroxybutyl-(S)-3-hydroxybutanoate, (S)-3-hydroxybutyl-(R)-3-hydroxybutanoate, (S)-3-hydroxybutyl-(S)-3-hydroxybutanoate, or the pharmaceutically acceptable salt thereof.

16. The method of claim 1, wherein the compound has structure Ill or the pharmaceutically acceptable salt thereof 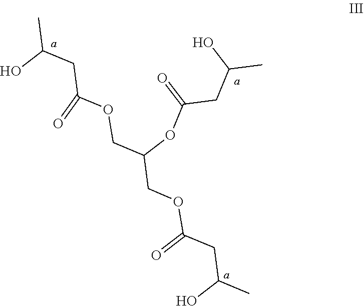 [SMILES: CC(O)CC(=O)OCC(COC(=O)CC(C)O)OC(=O)CC(C)O]
wherein the stereochemistry at carbon a is substantially S or racemic.

17. The method of claim 1, wherein the aneurysm is an abdominal aortic aneurysm, a thoracic aortic aneurysm, or a cerebral aneurysm.

18. The method of claim 1, wherein the dissection in the blood vessel is an abdominal aortic dissection or a thoracic aortic dissection.

19. The method of claim 1, wherein the compound further treats or prevents atherosclerosis.

20. The method of claim 1, wherein the compound provides one of the following: reduces the size of aneurysmal plaque in the subject; reduces the inner diameter, exterior diameter, or a combination thereof of the aneurysm in the subject; recovers the stiffness of the vessel wall of the aneurysm; converts vascular smooth muscle cells into myofibroblasts; removes senescent cells from the vessel of the subject; prevents the formation of senescent cells in a vessel; or converts senescent cells to non-senescent cells.

21. (canceled)

22. (canceled)

23. (canceled)

24. (canceled)

25. (canceled)

26. (canceled)

27. The method of claim 1, wherein the compound is administered as a pharmaceutical composition.

28. The method of claim 1, wherein the compound is administered orally to the subject, intravenously, intramuscularly, subcutaneously, or intra-articularly to the subject, or the compound is administered to the subject by a stent.

29. (canceled)

30. (canceled)

31. (canceled)

32. The method of claim 1, wherein the subject is furthered administered an effective amount of a KYNU inhibitor, a KMO inhibitor, a 3-HAO upregulator, or acipimox, or a pharmaceutically acceptable salt thereof.

33. The method of claim 1, wherein the subject is further undergoing treatment with a ketogenic diet, a ketone-ester, or a combination thereof.

34. The method of claim 1, wherein the subject has an underlying medical disorder comprising Marfan syndrome, Loeys-Dietz syndrome, aneurysms-osteoarthritis syndrome, Ehlers-Danlos syndrome, familial thoracic aortic aneurysm/dissection, Shprintzen-Goldberg syndrome, cutis laxa syndrome, aortic valve disease, arterial tortuosity syndrome, X-linked Alport syndrome, Turner syndrome, or a congenital heart malformation.

35. The method of claim 1, wherein the compound treats or prevents an aneurysm or dissection in a subject having a bicuspid aortic valve (BAV).

36. The method of claim 1, wherein the subject is identified as at risk of having an aneurysm or dissection of a blood vessel.

37. The method of claim 36, wherein the subject is screened for a genetic mutation that predisposes the subject to an aneurysm, a dissection in a blood vessel, or a combination thereof.

38. The method of claim 37, wherein the subject has a mutated gene that encodes proteins involved in vascular smooth muscle cell contraction and adhesion to the extracellular matrix (ECM), transforming growth factor)-β signaling pathway, or smooth muscle cell metabolism.

39. The method of claim 37, wherein the subject has a mutated gene, wherein the gene comprises FBN1, lysyl oxidase (LOX), smooth muscle myosin heavy chain 11 (MYH11), smooth muscle α-actin 2 (ACTA2), myosin light chain kinase (MYLK), protein kinase cGMP-dependent type 1 (PRKG1), α-1 procollagen, type III (COL3A1), TGF-β receptor type II (TGFBR2), TGF-β receptor type I (TGFBR1), TGF-β2 (TGFB2), mothers against decapentaplegic drosophila homolog 3 (SMAD3), α-1 procollagen, type I (COL1A1), α-2 procollagen, type I (COL1A2), mediator complex subunit 12 (MED12), mothers against decapentaplegic drosophila homolog 4 (SMAD4), procollagen-lysine,2-oxoglutarate 5-dioxygenase 3 (PLOD3), endoglin (ENG), activin A receptor like Type 1 (ACVRL1), neurofibromatosis type 1 (NF1), or any combination thereof.

40. The method of claim 1, wherein the subject is screened for an elevated level of 3-hydroxyanthranilic acid (3-HAA).

41. The method of claim 40, wherein the method comprises
(a) determining the level of 3-hydroxyanthranilic acid (3-HAA) present in a sample from the subject;
(b) comparing the subject's level of 3-HAA to a range of standardized levels of 3-HAA derived from individuals without an aneurysm or dissection in a blood vessel (“normal range”); and
(c) administering the compound to subject if the subject's levels of 3-HAA is greater than the normal range.

42. A method of treating or preventing atherosclerosis in a subject comprising administering to the subject an effective amount of one or more compounds of structure I or the pharmaceutically acceptable salt thereof:
